SEQUENCE LISTING (12 sequences)

SEQ ID NO: 1 - DNA, 1002 bp, Homo sapiens
atgggtgactggggcttcctggagaagttgctggaccaggtccgagagcactcgaccgtggtgggtaagatctggctgacggtgctcttcatcttccgcatcctcatcctgggcctggccggcgagtcagtgtggggtgacgagcagtcagatttcgagtgtaacacggcccagccaggctgcaccaacgtctgctatgaccaggccttccccatctcccacatccgctactgggtgctgcagttcctcttcgtcagcacacccaccctggtctacctgggccatgtcatttacctgtctcggcgagaagagcggctggcgcagaaggagggggagctgcgggcactgccggccaaggacccacaggtggagcgggcgctggccggcatagagcttcagatggccaagatctcggtggcagaagatggtcgcctgcgcattccgcgagcactgatgggcacctatgtcgccagtgtgctctgcaagagtgtgctagaggcaggcttcctctatggccagtggcgcctgtacggctggaccatggagcccgtgtttgtgtgccagcgagcaccctgcccctacctcgtggactgctttgtctctcgccccacggagaagaccatcttcatcatcttcatgttggtggttggactcatctccctggtgcttaacctgctggagttggtgcacctgctgtgtcgctgcctcagccgggggatgagggcacggcaaggccaagacgcacccccgacccagggcacctcctcagacccttacacggaccagggtcttcttctacctccccgtggccaggggccctcatccccaccatgccccacctacaatgggctctcatccagtgagcagaactgggccaacctgaccacagaggagaggctggcgtcttccaggccccctctcttcctggacccaccccctcagaatggccaaaaacccccaagtcgtcccagcagctctgcttctaagaagcagtatgtatag

SEQ ID NO: 2 - protein, 333 aa, Homo sapiens
MGDWGFLEKLLDQVREHSTVVGKIWLTVLFIFRILILGLAGESVWGDEQSDFECNTAQPGCTNVCYDQAFPISHIRYWVLQFLFVSTPTLVYLGHVIYLSRREERLAQKEGELRALPAKDPQVERALAGIELQMAKISVAEDGRLRIPRALMGTYVASVLCKSVLEAGFLYGQWRLYGWTMEPVFVCQRAPCPYLVDCFVSRPTEKTIFIIFMLVVGLISLVLNLLELVHLLCRCLSRGMRARQGQDAPPTQGTSSDPYTDQGLLLPPRGQGPSSPPCPTYNGLSSSEQNWANLTTEERLASSRPPLFLDPPPQNGQKPPSRPSSSASKKQYV

SEQ ID NO: 3 - DNA, 1077 bp, Mus musculus
atgggtgactggagcttcctgggggagttcctggaggaggtccacaagcactccacagtcatcggcaaggtctggctcactgtcctgttcattttccgcatgctggtcctgggcaccgctgctgagtcctcctggggagatgagcaggccgacttccggtgcgataccattcagcctggttgccaaaatgtctgctatgaccaagccttccccatctcccacattcgttattgggtactgcagatcatctttgtgtccacgccttctctagtgtacatgggccatgccatgcacactgtgcgcatgcaggaaaagcagaaattgcgggatgctgagaaagctaaagaggcccaccgcactggtgcctatgagtacccagtagccgaaaaggccgagctgtcctgctggaaagaagtagatgggaagattgtcctccagggcaccctactcaacacctatgtctgcaccattctgatccgcaccaccatggaggtggccttcatcgtaggccagtacctcctctatgggatcttcctggataccctgcatgtctgccgcaggagtccctgtccccacccagtcaactgttatgtttcgaggcccacggagaagaatgtcttcattgtctttatgatggctgtggctggactgtctctgtttctcagcctggctgaactctaccacctgggctggaagaagatccgacagcgctttggcaagtcacggcagggtgtggacaagcaccagctgcctggccctcccaccagcctcgtccagagcctcactcctccccctgacttcaatcagtgcctaaagaacagctccggagagaaattcttcagcgacttcagtaataacatgggctcccggaagaatccagacgctctggccactggggaagtgccaaaccaggagcagattccaggggaaggcttcatccacatgcactatagccagaagccagagtacgccagtggagcctctgcgggccaccgccttcctcagggctaccatagtgacaaacggcgccttagtaaggccagcagcaaagcaaggtcagatgacctgtcagtgtga

SEQ ID NO: 4 - protein, 358 aa, Mus musculus
MGDWSFLGEFLEEVHKHSTVIGKVWLTVLFIFRMLVLGTAAESSWGDEQADFRCDTIQPGCQNVCYDQAFPISHIRYWVLQIIFVSTPSLVYMGHAMHTVRMQEKQKLRDAEKAKEAHRTGAYEYPVAEKAELSCWKEVDGKIVLQGTLLNTYVCTILIRTTMEVAFIVGQYLLYGIFLDTLHVCRRSPCPHPVNCYVSRPTEKNVFIVFMMAVAGLSLFLSLAELYHLGWKKIRQRFGKSRQGVDKHQLPGPPTSLVQSLTPPPDFNQCLKNSSGEKFFSDFSNNMGSRKNPDALATGEVPNQEQIPGEGFIHMHYSQKPEYASGASAGHRLPQGYHSDKRRLSKASSKARSDDLSV

SEQ ID NO: 5 - DNA, 1149 bp, Mus musculus
atgggtgattggagtgccttggggaagctgctggacaaggtccaagcctactccacggccggagggaaggtgtggctgtcggtgctcttcattttcagaatcctgctcctggggacagcggttgagtcagcttggggtgatgaacagtctgcctttcgctgtaacactcaacaacccggttgtgaaaatgtctgctatgacaagtccttccccatctctcacgtgcgcttctgggtccttcagatcatattcgtgtctgtgcccacactcctgtacttggctcacgtgttctatgtgatgagaaaggaagagaagctgaacaagaaagaagaggagctcaaagtggcgcagaccgacggggtcaacgtggagatgcacctgaagcagattgaaatcaagaagttcaagtatgggattgaagaacacggcaaggtgaagatgagaggtggcctgctgagaacctacatcatcagcatcctcttcaagtctgtcttcgaggtggccttcctgctgatccagtggtacatctatgggttcagcctgagtgcggtctacacctgcaagagagatccctgcccccaccaggtggactgcttcctctcacgtcccacggagaaaaccatcttcatcatcttcatgctggtggtgtccttggtgtctctcgctctgaatatcattgagctcttctatgtcttcttcaagggcgttaaggatcgcgtgaagggaagaagcgatccttaccacgccaccaccggcccactgagcccatccaaagactgcggatctccaaaatatgcttacttcaatggctgctcctcaccaacggccccactctcacctatgtctcctcctgggtacaagctggtcactggtgacagaaacaattcctcctgccgcaattacaacaagcaagccagcgagcaaaactgggcgaattacagcgcagagcaaaatcgaatggggcaggccggaagcaccatctccaactcccacgcccagccgtttgatttccctgacgacagccaaaatgccaaaaaagttgctgctggacacgaactccagcccttagctatcgtggatcagcgaccttccagcagagccagcagccgcgccagcagcagacctcggcctgatgacctggagatctaa

SEQ ID NO: 6 - protein, 382 aa, Mus musculus
MGDWSALGKLLDKVQAYSTAGGKVWLSVLFIFRILLLGTAVESAWGDEQSAFRCNTQQPGCENVCYDKSFPISHVRFWVLQIIFVSVPTLLYLAHVFYVMRKEEKLNKKEEELKVAQTDGVNVEMHLKQIEIKKFKYGIEEHGKVKMRGGLLRTYIISILFKSVFEVAFLLIQWYIYGFSLSAVYTCKRDPCPHQVDCFLSRPTEKTIFIIFMLVVSLVSLALNIIELFYVFFKGVKDRVKGRSDPYHATTGPLSPSKDCGSPKYAYFNGCSSPTAPLSPMSPPGYKLVTGDRNNSSCRNYNKQASEQNWANYSAEQNRMGQAGSTISNSHAQPFDFPDDSQNAKKVAAGHELQPLAIVDQRPSSRASSRASSRPRPDDLEI

SEQ ID NO: 7 - DNA, 1191 bp, Mus musculus
atgagttggagcttcctgactcgcctgctagaggagatccacaaccattcgacatttgtagggaagatctggctcactgtgctgattgtctttcgaattgtcctaactgctgtaggaggagagtccatctactatgatgaacaaagcaaatttgtgtgcaacacagagcagccgggctgtgagaatgtctgctatgatgcctttgccccgctctcccacgtgcgcttctgggtattccagatcatcctggttgcaactccctctgtgatgtacctgggatatgctattcataagattgccaaaatggagcatggtgaggcagacaagaaggcagctcggagcaaaccctatgccatgcgttggaaacagcaccgggctctggaagaaacggaagaggaccatgaagaggatcctatgatgtatccagagatggagttagaaagcgaaaaagaaaataaagagcagagccaaccaaaacctaagcatgatggccgacgacgaattcgagaggatgggctcatgaaaatctatgtgttgcagctgctggccaggactgtgtttgaggtgggctttctaatagggcagtatttcctgtatggcttccaagtccacccattttatgtgtgcagcagacttccttgccctcataagatagactgctttatttctagacccactgaaaagaccatcttccttctgataatgtatggtgtcacaggcctctgcctattgcttaacatttgggagatgcttcacttagggtttgggacaattcgagactcactaaacagtaaaaggagggaacttgatgatccgggtgcttataattatcctttcacttggaacacaccctctgctccccctggctataacattgctgtcaaaccagatcagatccagtacactgagctgtccaatgctaagattgcctacaagcaaaacaaagccaatattgcccaggaacagcagtacggcagccacgaggaacacctcccggctgatctggagactctgcagcgggagatcagaatggctcaggaacgattggacctagcaatccaggcctaccatcaccaaaacaacccccatggtcctcgggaaaagaaggccaaagtggggtccaaatctgggtccaacaaaagcagtattagtagcaaatcaggggatgggaagacctccgtctggatttaa

SEQ ID NO: 8 - protein, 396 aa, Mus musculus
MSWSFLTRLLEEIHNHSTFVGKIWLTVLIVFRIVLTAVGGESIYYDEQSKFVCNTEQPGCENVCYDAFAPLSHVRFWVFQIILVATPSVMYLGYAIHKIAKMEHGEADKKAARSKPYAMRWKQHRALEETEEDHEEDPMMYPEMELESEKENKEQSQPKPKHDGRRRIREDGLMKIYVLQLLARTVFEVGFLIGQYFLYGFQVHPFYVCSRLPCPHKIDCFISRPTEKTIFLLIMYGVTGLCLLLNIWEMLHLGFGTIRDSLNSKRRELDDPGAYNYPFTWNTPSAPPGYNIAVKPDQIQYTELSNAKIAYKQNKANIAQEQQYGSHEEHLPADLETLQREIRMAQERLDLAIQAYHHQNNPHGPREKKAKVGSKSGSNKSSISSKSGDGKTSVWI

SEQ ID NO: 9 - DNA, 24 bp, Artificial Sequence
  FLAG epitope
gactacaaagacgatgacgacaag

SEQ ID NO: 10 - protein, 8 aa, Artificial Sequence
  FLAG epitope
DYKDDDDK

SEQ ID NO: 11 - DNA, 27 bp, Artificial Sequence
  HA epitope
tacccatacgacgtcccagactacgct

SEQ ID NO: 12 - protein, 9 aa, Artificial Sequence
  HA epitope
YPYDVPDYA

(19) United States
(12) Patent Application Publication
(10) Pub. No.: US 2003/0215424 A1
(43) Pub. Date: Nov. 20, 2003

(54) Method of modulating angiogenesis

(76) Inventors: Kyung Hwan Seul, Olympia Fields, IL (US); Keum Yee Kang, Olympia Fields, IL (US); Eric C. Beyer, Chicago, IL (US)

Correspondence Address: JHK Law, P.O. Box 1078, La Canada, CA, 91012-1078, US

(21) Appl. No.: 10/438,537

(22) Filed: May 15, 2003

Related U.S. Application Data
(60) Provisional application No. 60/380,947, filed on May 15, 2002.

(51) Int. Cl. A61K 48/00; A61K 38/17

(52) U.S. Cl. 424/93.2; 514/12; 514/44

(57) ABSTRACT

Disclosed are methods for regulating angiogenesis using gap junctions. Regulator molecules are vascular connexins. The methods comprise administering to the animals one or more connexin recombinant viruses containing connexin genes. Modulating angiogenesis includes the inhibition or induction of angiogenesis.

### CROSS-REFERENCE To RELATED APPLICATIONS

The present application claims the benefit of priority to U.S. Provisional Application No. 60/380,947, filed May 15, 2002, which is incorporated by reference herein in its entirety.

### BACKGROUND OF THE INVENTION

1. Field of the Invention

This invention pertains to a method of modulating angiogenesis by administering gap junction polypeptides to a subject. The present invention also relates to a method of stimulating or inhibiting proliferation or migration of endothelial or muscle cells by connecting the cells with gap junction polypeptides. The present invention is also directed to a method of treating diseases that are related to angiogenesis.

2. General Background and State of the Art

Angiogenesis is the process of growth of new capillaries from pre-existing blood vessels. In the adult mammal, the vasculature remains quiescent in most tissues, except for episodes of transient neovascularization as occur in the female reproductive system and wound repair (Hyder and Stancel, 1999). Angiogenesis is involved in the development and progression of a variety of disorders, including diabetic retinopathy, psoriasis, rheumatoid arthritis, cardiovascular diseases, and tumor growth (Folkman, 1995; Liekens et al., 2001).

Endogenous inhibitors may influence one or several steps of angiogenesis. They may antagonize angiogenic activity induced by growth factors or inhibit the proteolytic activity of angiogenic proteinases, endothelial cell proliferation, migration, or microtube formation. Once regulators of angiogenesis such as cytokines from surrounding local environment contact cell surfaces of the endothelial cells, they activate intracellular signaling pathways and change the type and/or concentration of intracellular signaling molecules.

The growth of new blood vessels requires quiescent endothelial cells to degrade the local basement membrane, to change its morphology, to proliferate, to migrate, to invade into the surrounding stromal tissue, to form microtubes to sprout new capillaries, and to reconstitute new basement membrane (Thompson, 1999; Liekens et al., 2001). The complex process of angiogenesis implies the presence of multiple controls, which can be switched on and off within a short period.

Over the years, researchers have focused on treating a single type of diseased cell or modifying one particular step in the complex process of angiogenesis to treat angiogenesis related diseases. Conventionally known molecules such as VEGF, angiostatin, endostatin, interferons, thrombospondin, tissue inhibitors of metalloproteinases, or other modifying ligands, antibodies, soluble receptors, antisense molecules, dominant-negative mutants of receptors and the like modify a specific point in the complex intracellular and extracellular signaling cascade of individual cells. The treatment generally works under specific conditions, but fails when target cells or even normal cells begin to adapt to the loaded therapeutics. Another reason for the lack of success in treating angiogenesis-related diseases relates to the heterogeneity of human vascular beds. Angiogenesis related diseases have been difficult to treat because each vascular bed embraces different types of tissues, which means their response to angiogenic modulators may be different.

Whether angiogenesis related diseases occur in specific organs such as skin wound injury or rheumatoid arthritis of joints, or whole body such as metastasized tumors, there are extensive adaptive processes in their local environment composed of normal cells and diseased cells. Interactions between them as well as those within themselves are important in the healing process. Accordingly, we thought that modulating the group behavior of vascular cells might provide an efficient way to control angiogenesis. Gap junction channel proteins may be used for this purpose.

Gap junctions that directly connect the cytoplasm of neighboring cells, in contrast to surface channels that transmit signals from outside to inside, transfer intracellular molecules between cells by making aqueous channels. They are responsible for synchronous behavior of grouped cells in local environments.

Gap junctions are of central importance in the growth and differentiation of cells. Gap junction channels are formed from two hemichannels each located within the cell membrane of two adjacent cells (reviewed in Beyer et al., 1997; Beyer and Willecke, 2000). Each hemi-channel is made up of six subunit proteins (connexins, Cx) surrounding a pore that permits substances to pass between the cells without entering the extracellular fluid (FIG. 1). The diameter of the channel is about 2 nm, and permits the rapid intercellular exchange of small molecules up to about 1000 Da (FIG. 2). Gap junction channels participate in the regulation of diverse functions, including controlling cell growth, facilitating pattern formation during development, coordinating contraction of smooth and cardiac muscle cells, transmitting neuronal signals at electrotonic synapses, and synchronizing endocrine and exocrine secretion. Twenty-one different connexins are present in the human or mouse genome; these connexins show different patterns of tissue distribution, developmental expression, and channel characteristics (FIG. 3). Blood vessels express four different subunit gap junction proteins or connexins: Cx37, Cx40, Cx43, and Cx45 (Davis et al., 1994; Beyer et al., 1997; Seul and Beyer, 2000). Each connexin has different permeability and selectivity characteristics for ions and other intracellular molecules and different responses to external stimuli such as growth factors and vasoactive agents.

In general, adult tissue cells use more than one type of connexin to exchange intracellular signaling molecules with neighboring cells through gap junction channels. They regulate the ratio of their connexin types, or turn one of them on or off for adaptation when the external environment changes. This modulation of connexin ratios results in changes in transfer of intracellular signaling molecules through gap junction channels between cells, because different gap junction channels act as filters for small molecules (through their different preferences regarding molecular size, surface charges, etc). During the early stages of development, the cells of the vascular system may use these different gap junction channel characteristics as regulators of cell growth and differentiation along the course of vessel formation.

Recent experiments have suggested a role for connexins in growth and coordinated migration of vascular wall cells and recovery from wounding. Cx37 and Cx43 are regulated differentially by cell density, growth, and TGF-β1 in cultured bovine aortic endothelial cells (Larson et al., 1997). Both Cx37 and Cx43 are increased in regenerating endothelium after vessel injury (Yeh et al., 2000). Using the PymT-transformed mouse endothelial cell lines, Kwak et al., (2001) found that mechanical wounding increased expression of Cx43 and decreased expression of Cx37 at the site of injury. Transcripts of Cx43 are decreased and those of Cx37 are increased by shear stress in cultured HUVECs (McCormick et al., 2001). Gap junctional communication of smooth muscle cells has also been recognized for a long time, but the exact role and regulation of connexins in these cells remain unknown. Cx43 between smooth muscle cells is upregulated after balloon catheter injury in the rat carotid artery (Yeh et al., 1997) and during early stages of human coronary atherosclerosis (Blackburn, 1995). Cx37, but not Cx40 or Cx43, is induced in vascular smooth muscle cells during coronary arteriogenesis (Cai et al, 2001).

Gap junctions of blood vessels have long been considered to act as important modulators of vascular function including systemic blood pressure and local vasomotor responses. In vitro experiments have shown that NO, EDHF (endothelium-derived hyperpolarizing factor) and intracellular ions might propagate through gap junctions (Edwards et al., 1999; Liao et al., 2001). Recently, it has been suggested that Cx40 is required for normal transmission of an endothelium-dependent vasodilator response in Cx40-deficient mice (de Wit et al., 2000). An endothelial cell-specific knockout of Cx43 causes hypotension and bradycardia in mice (Liao et al., 2001). The Cx45 knock-out and double knockout animals for Cx37 and Cx40 are embryonic lethal, and they show defects in vessel formation.

Although studies in many systems (including normal and transformed cells and transgenic and knockout animals) have suggested an important relationship between gap junction mediated intercellular commnunication and cell growth of blood vessels, definitive characterization of the role of connexins has been lacking. This deficit has been due to the lack of an appropriate system for delivery of connexins into adult tissues, and also by the non-viability of adult knock-out animals for the vascular connexins.

There still exists a need for improved reagents that reduce angiogenesis while overcoming the shortcomings of known reagents for modulating angiogenesis.

### SUMMARY OF THE INVENTION

The invention provides methods and reagents for regulating angiogenesis and allows for the treatment of various angiogenesis-associated conditions.

We have made viruses, and in particular adenoviruses, that can deliver gap junction proteins to endothelial cells of adult blood vessels. Using this system, we have found that connexins regulate angiogenesis of blood vessels by regulating the growth and death of vascular endothelial and smooth muscle cells. Such novel reagents and methods for using them are useful for treating conditions associated with angiogenesis including, without limitation, neoplasia, rheumatoid arthritis, endometriosis, psoriasis, vascular retinopathies, and remodeling of injured tissues.

In accordance with the present invention, compositions and methods are provided that are effective for modulating angiogenesis, and inhibiting unwanted angiogenesis, preferably with Cx37 and promoting angiogenesis, preferably with Cx43. Combinations of each vascular connexins provide new concepts of methods of controlling angiogenesis of different vascular beds, because endothelial cells of different organs communicate with each other by using different sets of vascular connexins. The present invention includes proteins, which has been named “connexins”, defined by their ability to modulate the intercellular transfer of intracellular signaling molecules generated by angiogenic or antiangiogenic factors. Vascular connexins may include without limitation Cx37, Cx40, Cx43, and Cx45.

The amino acid sequences of connexins vary slightly between species. It is to be understood that the number of amino acids in connexin molecules may vary and all amino acid sequences that have angiogenesis modulating activity are contemplated as being included in the present invention.

The present invention provides methods and compositions for treating diseases and processes mediated by undesired and uncontrolled angiogenesis by administering to a human or animal a composition comprising substantially connexins in a dosage sufficient to inhibit or promote angiogenesis.

Cx37 is particularly useful for treating, or for repressing the growth of, tumors. Administration of Cx37 to a human or animal with prevascularized metastasized tumors prevents the growth or expansion of those tumors. Combinations of other connexins (Cx43, Cx40 and Cx45) with Cx37 modify the antiangiogenetic effects of Cx37.

The present invention also includes mutants of connexins. In one embodiment, mutants include those in which protein kinase A (PKA), protein kinase C (PKC), or casein kinase (CK) sensitive phosphorylation sites are deleted or mutated. Some tumor cells activate PKA, PKC, or CK activity to grow, and activated PKA, PKC, or CK inhibits connexin channels. Mutants of connexins further include those which are deleted or mutated at the serine/threonine amino acid of the cytoplasmic tail, and those in which the cytoplasmic tail is truncated.

Cx43 promotes anglogenesis by enhancing endothelial growth and migration from wounded vessels. Cx43 is also useful for preventing restenosis after angioplasty of atherosclerosis. It is also useful for tissue remodeling of injured tissues such as skin injury, bone fracture and myocardial infarction.

It is another object of the present invention to provide a method of therapeutic antiangiogenesis and composition for treating diseases and processes that are mediated by angiogenesis including, but not limited to, hemangioma, solid tumors, blood borne tumors, leukemia, mietastasis, telangiectasia, psoriasis, scleroderma, pyogenic granuloma, myocardial angiogenesis, Crohn's disease, plaque neovascularization, coronary collaterals, cerebral collaterals, arteriovenous malformations, ischemic limb angiogenesis, corneal diseases, rubeosis, neovascular glaucoma, diabetic retinopathy, retrolental fibroplasia, arthritis, diabetic neovasculauization, macular degeneration, wound healing, peptic ulcer, Helicobacter related diseases, fractures, keloids, vasculogenesis, hematopoiesis, ovulation, menstruation, placentation, and cat scratch fever.

It is yet another object of the present invention to provide a method of therapeutic angiogenesis and composition for treating diseases and processes that are mediated by angiogenesis including, but not limited to, restenosis after angioplasty, ischemic coronary artery disease, congestive heart failure, critical limb ischemia, and gastroduodenal ulcer.

It is yet another object of the present invention to provide a therapy for cancer that has minimal side effects, including toxic side effects.

Another object of the present invention is to provide a method for targeted delivery of connexin-related compositions to specific locations.

Yet another object of the invention is to provide compositions and methods useful for gene therapy for the modulation of angiogenic processes.

The present invention is directed to a method of modulating angiogenesis comprising administering a gap junction polypeptide to endothelial cells. In this method, the gap junction polypeptide is vascular connexin polypeptide. The connexin is connexin37 (Cx37), connexin4O (Cx40), connexin43 (Cx43), connexin45 (Cx45) or combination thereof

The present invention is also directed to a method of modulating angiogenesis comprising:

a) generating a recombinant viral or plasmid vector comprising a DNA sequence encoding a member of a connexin family of polypeptides operatively linked to a promoter;

b) transfecting in vitro a population of cultured cells with said recombinant vector, resulting in a population of transfected cells; and

c) transplanting said transfected cells to a mammalian host, such that expression of said DNA sequence within the mammal results in modulating angiogenesis.

The present invention is also directed to a method of inhibiting proliferation of endothelial cells comprising administering to the endothelial cells connexin37 polypeptide or a variant thereof.

The present invention is also directed to a method of inhibiting growth or proliferation of endothelial cells, comprising:

a) generating a recombinant viral or plasmid vector comprising a DNA sequence encoding a member of a connexin family of polypeptides operatively linked to a promoter;

b) transfecting iii vitro a population of cultured cells with the recombinant vector, resulting in a population of transfected cells; and

c) transplanting said transfected cells to a mammalian host, such that expression of said DNA sequence within the mammal results in inhibition of endothelial cell growth or proliferation.

In this method, the vector may be without limitation a viral vector or a plasmid vector. The connexin may be connexin37.

The present invention is also directed to a method of promoting growth or proliferation of endothelial cells, comprising administering to the endothelial cells a connexin polypeptide or a variant thereof. In this method, the connexin polypeptide may be connexin40, connexin43 or connexin45 polypeptide or a variant thereof.

The present invention is also directed to a method of promoting growth or proliferation of endothelial cells, comprising:

a) generating a recombinant viral or plasmid vector comprising a DNA sequence encoding a member of a connexin family of polypeptides operatively linked to a promoter;

b) transfecting in vitro a population of cultured cells with said recombinant vector, resulting in a population of transfected cells; and

c) transplanting said transfected cells to a mammalian host, such that expression of said DNA sequence within the mammal results in promotion of endothelial cell growth or proliferation. In this method, the vector may be without limitation a viral vector or a plasmid vector. The connexin may be connexin40, connexin43, connexin45 or a combination thereof.

In addition, the present invention is also directed to a method of treating angiogenesis related disease comprising administering to a mammal in need thereof a therapeutically effective amount of a gap junction polypeptide. In this method, the gap junction polypeptide is connexin37 (Cx37), connexin40 (Cx40), connexin43 (Cx43), connexin45 (Cx45) or a combination thereof. The angiogenesis related disease is solid tumors, blood born tumors, tumor metastasis, benign tumors, rheumatoid arthritis; psoriasis; ocular angiogenic diseases, Osler-Webber Syndrome, myocardial angiogenesis, plaque neovascularization, telangiectasia, hemophiliac joints, angiofibroma, wound granulation, intestinal adhesions, Crohn's disease, atherosclerosis, scleroderma, hypertrophic scars.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIGS. 1A-1C show topological (A) and structural (B) model of connexin orientation within the junctional plasma membrane. Cytoplasmic loop (CL) and tail (CT) correspond to unique, connexin-specific regions. Structural model of gap junction plaque (B) based on x-ray diffraction and electron microscopy studies of isolated rodent liver gap junctions. (C) Immunofluorescent localization of Cx40 between two cells. Cultured HeLa cells were incubated with Cx40-adenovirus for 24 h, cells were fixed and stained to detect Cx40 immunoreactivity. Intercellular membrane junctions of paired two cells show abundant expression of gap junction molecules (*).

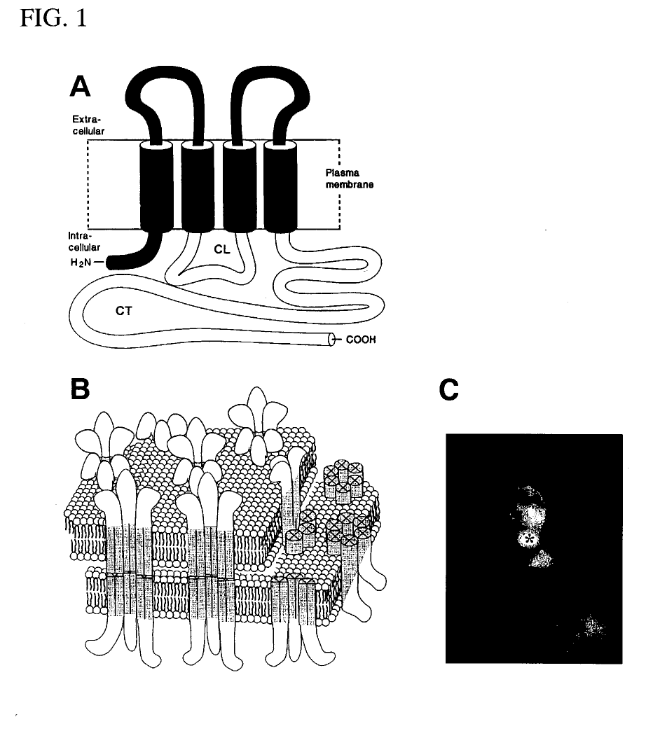

FIG. 2 shows transfer of dye (Lucifer Yellow) through gap junctions in normal rat kidney cells. Lucifer Yellow (Mr=443, valence=−2, ≈10 A° diameter) was microinjected into single cell (*) and its transfer into adjacent cells was visualized by fluorescence inverted microscopy.

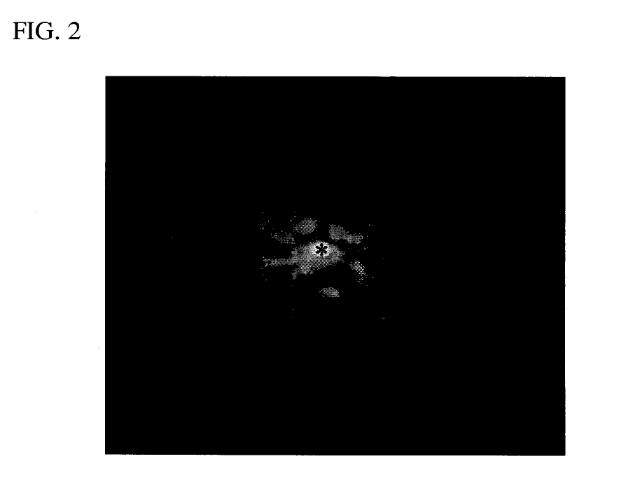

FIGS. 3A-3D show immunofluorescent detection of connexin40 in mouse kidney showing glomeruli (g) and their arterioles. A and D show longitudinal sections of rather long afferent arterioles, B shows a shorter arteriole, and C shows a arteriole in transverse section. Typical endothelial gap junction signals were clearly detected in longitudinally (top of the panel A) and transversely (top of the panel B) sectioned interlobular arterioles. Intense signals were detected at the juxtaglomerular apparatus (j) which is shown at different angles in longitudinal (A, B and D) and transverse (C) sections. All tissue sections shown were 40 μm-thick, except B (10 μm).

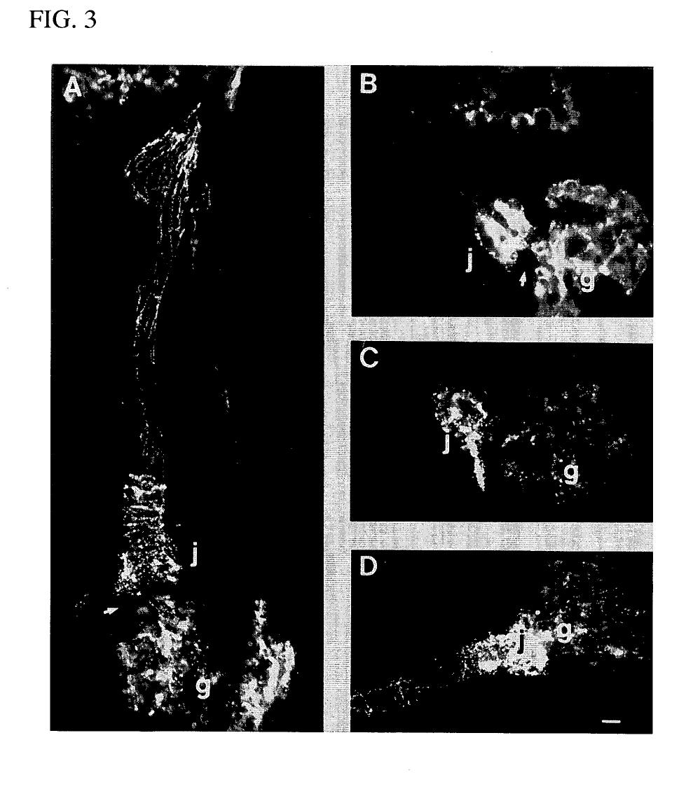

FIGS. 4A-4D show connexin-adenovirus treatment showing abundant connexin staining between cells both in vitro and in vivo. (A) Cx37 produced by Cx37 adenovirus infection of NRK cells was detected using anti-FLAG antibody. (B) HUVECs were infected with Cx40 adenovirus and reacted with anti-Cx40 antibody. (C) Cx43-adenovirus injected through tail vein of the adult BALB/C mouse leads to abundant expression of Cx43 in the vascular endothelial cells of the kidney. (D) This panel shows the low level of non-specific antibody binding. This mouse was infected with an adenovirus containing the CMV promoter alone, with no connexin insert, and reacted with anti-Cx43 antibodies followed by Cy3-conjugated secondary antibodies.

FIGS. 5A-5B show delivery of connexins by adenovirus into adult endothelial cells. (A) Immunohistochemistry. Endothelial cells endogenously express Cx43 as shown in the top panel. In the lower panel, HUVECs similarly infected with a Cx37, Cx43, or Cx40 adenovirus show abundant connexin staining between cells. (B) Western blotting. Adenoviral expression of Cx37 showed dose-dependency and down-regulated endogenous Cx43 expression.

FIGS. 6A-6B show effects of connexins on cell growth and viability. (A) Endothelial connexins have strongest effect on endothelial cells (*, P<0.001). Each connexin group was compared with control group. (B) Cx37-induced endothelial cell death was dose-dependent. Actively dividing cells were more susceptible than confluent cells. Cells were seeded into 24 well culture dishes by different densities; 5×10⁴ cells/well for low-confluency and 2×10⁶ cells/well for high-confluency experiments. Low-confluent cells were infected with viruses when they reached about 50% confluency after overnight culture.

FIGS. 7A-7C show modification of Cx37-induced endothelial cell death. (A) To test whether death signals induced by Cx37 spreads through extracellular or intracellular route, we treated normal HUVEC with conditioned media from cells treated with Cx37 or control virus (2.6×10⁸ pfu). Whole supernatants including cell debris from each well were collected, and transferred to new wells of HUVEC, and incubated for 2 hours. After washing with PBS, cells were cultured 2 more days with new media until viabilities were tested. HUVEC was not killed when supernatants of cells dead by Cx37 adenovirus applied to extracellular space of cells. This shows that death process does not spread through extracellular route, indicating that gap junction channels between cells may act as routes for spreading of death signals. (B) Gap junction blocker (carbenoxolone, CBX) potentiates Cx37-induced cell death (*, P<0.001). (C) Overexpression of Cx43 potentiates Cx37-induced death in HUVEC. Protein expression of Cx37 and Cx43 of this protocol was shown at top of (C) (*, P<0.001).

FIGS. 8A-8C show that Cx37-induced endothelial cell death is mediated by apoptosis. (A) Annexin V and propidium iodide (PI) staining of HUVECs. Apoptotic cells were visualized with Annexin V (green) and necrotic cells with PI (red). (B) Caspase 3 was increased in Cx37 adenovirus treated cells (*, P<0.01; n=6). (C) Time course of apoptosis visualized with the TUNEL assay. Scale bar, 10 μm.

FIGS. 9A-9D show influence of connexin expression on recovery from wounding. (A) Control-Ad. (B) Cx40-Ad treated cells result in growth of cells back across the wounds. (C) Cx37-Ad treated cells result in completely blocked growth of cells back across the wounds. (D) The growth was accelerated with Cx43 virus. Experiments were repeated 3 times. Underlying bars indicate original denuded areas.

FIGS. 10A-10C show that Cx37 virus blocks VEGF-induced angiogenesis within Matrigel in vivo. (A) Angiogenesis quantitated by hemoglobin content within Matrigel plug was significantly lower in Cx37 group than in control group (*, P<0.001; n=4-6; top). (B, C) Sections of Matrigel plugs stained with Masson's Trichrome shows that Cx37 virus completely blocks VEGF-induced angiogenesis in vivo (bottom). M, Matrigel; S, skeletal muscles. Matrigels from more than 6 animals were observed for each group.

FIG. 11 shows Cx37-Ad blocks angiogenesis following systemic delivery. Angiogenesis within Matrigel plugs was quantitated by measuring hemoglobin (Hb) concentration with Drabkin's kit (Sigma). Mice were systemically injected with 5×10⁸ pfu of Cx37 or control adenovirus through tail vein. After 2 days, Matrigel containing VEGF and heparin, but not adenovirus, was injected subcutaneously. The implanted gels were harvested 7 days after injection, and hemoglobin content was analyzed. The data are expressed as the mean±SE and significance was determined by Student's t test, *P<0.01.

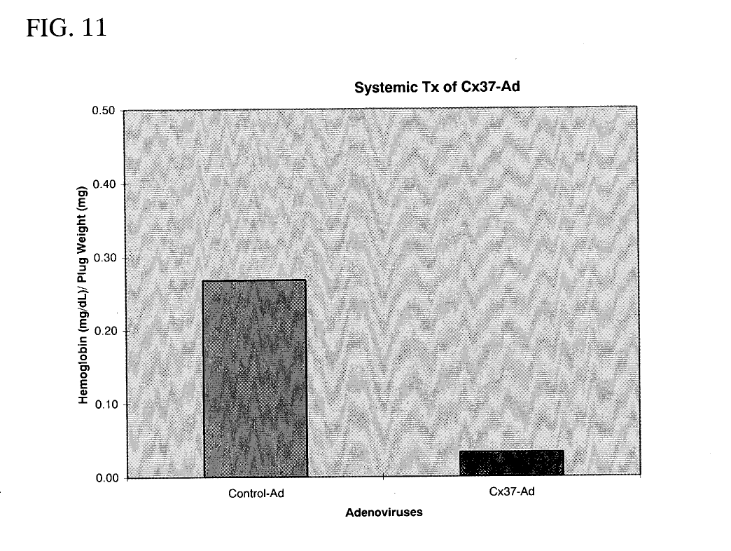

FIG. 12 shows that effects of connexins on endothelial cell growth differ according to cell growth stage. Two sets of 24 well culture dishes were plated with HUVECs. 4×10⁷ pfu of Cx-adenovirus was applied to cells when cultures became 100% confluent. After 2 days of incubation, one set of cells was split and replated into culture dishes at different ratios (50%, 25%, and 12.5%). One set of cells was continued at confluency without splitting. After further three days of incubation, cell proliferation was determined by XTT assay.

### DETAILED DESCRIPTION OF THE PREFERRED EMBODIMENTS

In the present application, “a” and “an” are used to refer to both single and a plurality of objects.

As used herein, “about” or “substantially” generally provides a leeway from being limited to an exact number. For example, as used in the context of the length of a polypeptide sequence, “about” or “substantially” indicates that the polypeptide is not to be limited to the recited number of amino acids. A few amino acids added to or subtracted from the N-terminus or C-terminus may be included so long as the functional activity such as its binding activity is present.

As used herein, administration “in combination with” one or more further therapeutic agents includes simultaneous (concurrent) and consecutive administration in any order.

As used herein, “amino acid” and “amino acids” refer to all naturally occurring L-α-amino acids. This definition is meant to include norleucine, ornithine, and homocysteine.

As used herein, in general, the term “amino acid sequence variant” refers to molecules with some differences in their amino acid sequences as compared to a reference (e.g. native sequence) polypeptide. The amino acid alterations may be substitutions, insertions, deletions or any desired combinations of such changes in a native amino acid sequence.

Substitutional variants are those that have at least one amino acid residue in a native sequence removed and a different amino acid inserted in its place at the same position. The substitutions may be single, where only one amino acid in the molecule has been substituted, or they may be multiple, where two or more amino acids have been substituted in the same molecule.

Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Also included within the scope of the invention are proteins or fragments or derivatives thereof which exhibit the same or similar biological activity and derivatives which are differentially modified during or after translation, e.g., by glycosylation, proteolytic cleavage, linkage to an antibody molecule or other cellular ligand, and so on.

Insertional variants are those with one or more amino acids inserted immediately adjacent to an amino acid at a particular position in a native amino acid sequence. Immediately adjacent to an amino acid means connected to either the α-carboxy or x-amino functional group of the amino acid.

Deletional variants are those with one or more amino acids in the native amino acid sequence removed. Ordinarily, deletional variants will have one or two amino acids deleted in a particular region of the molecule.

In one aspect, the polypeptide variants of the present invention may contain any number of amino acids or alterations of amino acids in the gap junction polypeptide, including substitutions and/or insertions and/or deletions in any region of the polypeptide molecule. In particular, the polypeptide variant includes a sequence that is at least about 70%, 75%, 80%, 85%, 90%, 95%, 96%, 97%, 98% or 99% identical to the polypeptide sequence represented by SEQ ID NOS: 2, 4, 6, or 8 and the presence of the variations do not hinder the pore forming or angiogenesis modulating function, including endothelial and/or muscle cell promotion or inhibition activity of the corresponding native polypeptide. In particular, such variations may include without limitation, deletions and mutations of protein kinase A, protein kinase C or casein kinase sensitive phosphorylation sites. Further variations may include those connexins which are deleted or mutated at their serine/threonine amino acid of the cytoplasmic tail, and in particular those in which the cytoplasmic tail is truncated.

As used herein, “angiogenesis” is meant the growth of a new blood vessel in which the proliferation and/or migration of an endothelial cell is a key step. By “inhibiting angiogenesis” is meant the inhibition of any of the steps of the process of angiogenesis that includes, without limitation, proliferation and/or migration of endothelial cells. By “promoting angiogenesis” is meant the promotion of any of the steps of the process of angiogenesis that includes, without limitation, proliferation and/or migration of endothelial cells.

As used herein, “angiogenesis modulation” refers to the inhibition and/or stimulation of endothelial and/or muscle cells, in particular vascular endothelial cells or smooth muscle cells, which include proliferation/growth or inhibition of such cells, and results in the control, regulation or remodeling of the formation of blood vessels. Administration of a combination of the gap junction polypeptides of the invention may result in the modulation of angiogenesis tailored to the particular condition.

As used herein, “angiogenesis related disease” refers to those diseases that are caused by either the proliferation of blood vessels or inhibition of formation of blood vessels.

As used herein, administration “in combination with” one or more further therapeutic agents includes simultaneous (concurrent) and consecutive administration in any order.

As used herein, “connexin family” of proteins refers to family of gap junction proteins that makes channels between connecting cells through which direct intercellular communication via diffusion of small molecules such as but not limited to ions, second messengers and metabolites. The connexin family consists of at least 20 members in human and 19 members in rodents.

As used herein, “gap junction polypeptide” or “connexin polypeptide” refers to a polypeptide that forms or participates in pore formation and transport of substances through the pore. Preferably, in a specific embodiment of the invention, the gap junction polypeptide may be a connexin polypeptide, including without limitation, connexins 37, 40, 43 or 45. In another aspect of the invention, the connexin polypeptide may have at least about 70%, 75%, 80%, 85%, 90%, 95%, 96%, 97%, 98%, 99% or 100% identity to the polypeptide sequence represented by SEQ ID NOS:2, 4, 6, or 8. In particular, gap junction polypeptide such as connexin 37 may inhibit the growth, proliferation or migration of endothelial and/or muscle cells. Gap junction polypeptide such as connexin 40, connexin 43 polypeptide may promote the growth of endothelial cells. In addition, a connexin polypeptide or a combination of connexin polypeptides may be used to modulate, regulate, or control the level of angiogenesis. Moreover, one connexin molecule may be used to potentiate the activity of another connexin molecule. For instance, without limitation, addition of connexin43 may potentiate the cell death inducing capability of connexin37.

As used herein, “gap junction polypeptide” refers to a polypeptide that is derived from a gap junction protein, but which is not limited to the specific sequence of the native form. It is understood that various mutations and conservative amino acid changes are tolerable, as well as certain non-conservative amino acid changes, so long as the polypeptide forms or participates in the formation of intercellular pores, and substances may be transported through the pores. Fragments and certain glycosylations are also permitted, indeed any change at all to the gap junction polypeptide is permitted so long as the angiogenesis modulation function is retained.

Applicants for the first time discovered that gap junction polypeptides may be used to remodel, regulate, control, or modulate angiogenesis by controlling the growth or inhibition of vascular endothelial cells and/or smooth muscle cells, and thus it would be within the purview of a person of skill in the art to make certain variations to the sequence, which retains the capability of these gap junction polypeptides to modulate angiogenesis.

As used herein, the term “capable of hybridizing under high stringency conditions” means annealing a strand of DNA complementary to the DNA of interest under highly stringent conditions. Likewise, “capable of hybridizing under low stringency conditions” refers to annealing a strand of DNA complementary to the DNA of interest under low stringency conditions. “High stringency conditions” for the annealing process may involve, for example, high temperature and/or low salt content, which disfavor hydrogen-bonding contacts among mismatched base pairs. “Low stringency conditions” would involve lower temperature, and/or higher salt concentration than that of high stringency conditions. Such conditions allow for two DNA strands to anneal if substantial, though not near complete complementarity exists between the two strands, as is the case among DNA strands that code for the same protein but differ in sequence due to the degeneracy of the genetic code. Appropriate stringency conditions which promote DNA hybridization, for example, 6×SSC at about 45° C., followed by a wash of 2×SSC at 50° C. are known to those skilled in the art or can be found in Current Protocols in Molecular Biology, John Wiley & Sons, N.Y. (1989), 6.31-6.3.6. For example, the salt concentration in the wash step can be selected from a low stringency of about 2×SSC at 50° C. to a high stringency of about 0.2×SSC at 50° C. In addition, the temperature in the wash step can be increased from low stringency at room temperature, about 22° C., to high stringency conditions, at about 75° C. Other stringency parameters are described in Maniatis, T., et al., Molecular Cloning: A Laboratory Manual, Cold Spring Harbor Laboratory Press, Cold Spring N.Y., (1982), at pp. 387-389; see also Sambrook J. et al., Molecular Cloning: A Laboratory Manual, Second Edition, Volume 2, Cold Spring Harbor Laboratory Press, Cold Spring, N.Y. at pp. 8.46-8.47 (1989).

As used herein, “carriers” include pharmaceutically acceptable carriers, excipients, or stabilizers which are nontoxic to the cell or mammal being exposed thereto at the dosages and concentrations employed. Often the pharmaceutically acceptable carrier is an aqueous pH buffered solution. Examples of pharmaceutically acceptable carriers include without limitation buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN®, polyethylene glycol (PEG), and PLURONICS®.

As used herein, “covalent derivatives” include modifications of a native polypeptide or a fragment thereof with an organic proteinaceous or non-proteinaceous derivatizing agent, and post-translational modifications. Covalent modifications are traditionally introduced by reacting targeted amino acid residues with an organic derivatizing agent that is capable of reacting with selected sides or terminal residues, or by harnessing mechanisms of post-translational modifications that function in selected recombinant host cells. Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the gap junction polypeptides of the present invention. Other post-translational modifications include hydroxylation of proline and lysine, phosphorylation of hydroxyl groups of seryl, tyrosine or threonyl residues, methylation of the α-amino groups of lysine, arginine, and histidine side chains (T. E. Creighton, Proteins: Structure and Molecular Properties, W. H. Freeman & Co., San Francisco, pp. 79-86 (1983)).

As used herein, “effective amount” is an amount sufficient to effect beneficial or desired clinical or biochemical results. An effective amount can be administered one or more times. For purposes of this invention, an effective amount of an inhibitor or stimulator gap junction polypeptide is an amount that is sufficient to palliate, ameliorate, stabilize, reverse, slow or delay the progression of an angiogenesis-related disease. In a preferred embodiment of the invention, the “effective amount” is defined as an amount of compound capable of modulating angiogenesis or treating an angiogenesis-related disease. In yet another embodiment, the “effective amount” is defined as the endothelial and/or muscle cell growth inhibitor or stimulator effective amount of the gap junction polypeptide.

As used herein, “fragment” refers to a part of a polypeptide, which retains usable and functional characteristics. For example, as used within the context of the present invention, the polypeptide fragment has the function of forming or participating in the formation of pores in intercellular contact. The polypeptide fragment may further result in either inhibiting or stimulating growth, migration or proliferation of vascular endothelial cells, and may further modulate angiogenesis and treat angiogenesis-related diseases.

As used herein, “host cell” includes an individual cell or cell culture which can be or has been a recipient of a vector of this invention. Host cells include progeny of a single host cell, and the progeny may not necessarily be completely identical (in morphology or in total DNA complement) to the original parent cell due to natural, accidental, or deliberate mutation and/or change. A host cell includes cells transfected or infected in vivo with a vector comprising a polynucleotide encoding a gap junction polypeptide.

As used herein, “immunohistochemistry” refers to a method that measures level of specific protein in a variety of tissues.

As used herein, “immunioprecipitation” refers to a biological method that quantitatively measures expression level of a protein and also qualitatively the interaction between polypeptides.

As used herein, “inhibitor” refers to a molecule that inhibits the growth or proliferation of endothelial cells.

As used herein, “mammal” for purposes of treatment refers to any animal classified as a mammal, including humans, domestic and farm animals, and zoo, spolts, or pet animals, such as dogs, cats, cattle, horses, sheep, pigs, and so on. Preferably, the mammal is human.

As used herein, “purified” or “isolated” molecule refers to biological molecules that are removed from their natural environment and are isolated or separated and are free from other components with which they are naturally associated.

As used herein, “sequence identity”, is defined as the percentage of amino acid residues in a candidate sequence that are identical with the amino acid residues in a native polypeptide sequence after aligning the sequences and introducing gaps, if necessary, to achieve the maximum percent sequence identity, and not considering any conservative substitutions as part of the sequence identity. The % sequence identity values are generated by the NCBI BLAST2.0 software as defined by Altschul et al., (1997), “Gapped BLAST and PSI-BLAST: a new generation of protein database search programs”, Nucleic Acids Res., 25:3389-3402. The parameters are set to default values, with the exception of the Penalty for mismatch, which is set to −1.

As used herein, “subject” is a vertebrate, preferably a mammal, more preferably a human.

As used herein, “treatment” is an approach for obtaining beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” refers to both therapeutic treatment and prophylactic or preventative measures. Those in need of treatment include those already with the disorder as well as those in which the disorder is to be prevented. “Palliating” a disease means that the extent and/or undesirable clinical manifestations of a disease state are lessened and/or the time course of the progression is slowed or lengthened, as compared to a situation without treatment.

As used herein, “vector” means a carrier that can contain or associate with specific nucleic acid sequences, which functions to transport the specific nucleic acid sequences into a cell. Examples of vectors include plasmids and infective microorganisms such as viruses, or non-viral vectors such as ligand-DNA conjugates, liposomes, lipid-DNA complexes. It may be desirable that recombinant DNA molecule comprising connexin DNA sequences are operatively linked to an expression control sequence to form expression vectors capable of expressing connexins. The transfected cells may be cells derived from the patient's normal tissue, the patient's diseased tissue, or may be non-patient cells.

Angiogenesis Modulation

The inventive system regulates the final stages of signal transduction pathways. The system provides a set of connexin polypeptides from the same protein family to modulate angiogenesis. It is also recognized that different connexins have opposing actions on angiogenesis. This approach provides for significantly advantageous therapeutic agents for diseases related to angiogenesis.

The present invention provides a method of treating angiogenic disorder such as tumor and wound healing, by modulating intercellular communication (i.e., transfer of intracellular signals through gap junction channels).

Actions of different bioactive molecules on cells result in changes of different sets of intracellular signaling molecules. Spread of these molecules to neighboring cells through gap junction channels (i.e., intercellular communication) is essential to cells for the subsequent division, movement, and tissue remodeling. The angiogenic phenotype in a tissue is dependent upon the local balance between angiogenic factors and inhibitors. It is now believed that a blood vessel uses different types of gap junctions during vasculogenesis, angiogenesis and vessel regression. This indicates that endothelial cells use different gap junction channels in the control of their growth patterns, combined with angiogenic or antiangiogenic factors. Controlling angiogenesis at the level of “signal spread through gap junctions” rather than through extracellular signals should be more efficient for controlling angiogenesis. This strategy is based on the limited number of different intracellular signaling molecules as compared to the large number of extracellular factors that may affect angiogenesis. Moreover, many of the extracellular factors share common intracellular signaling molecules including calcium ions, cyclic AMP, and inositol triphosphate. These molecules are transferred through gap junction channels and are reported to trigger the cell suicide program (Yasui et al., 2000).

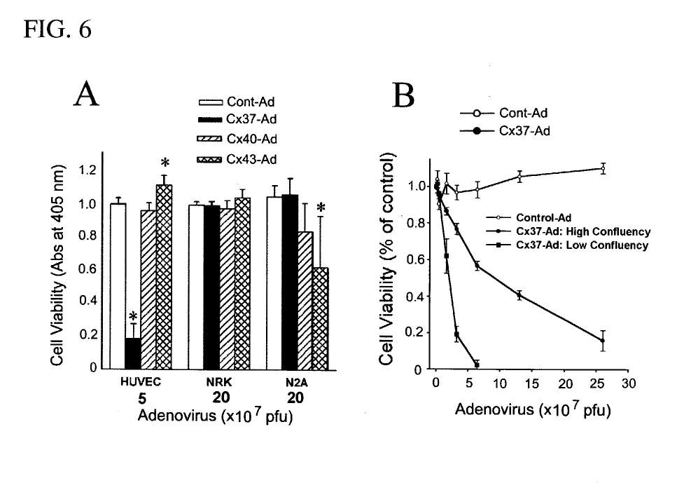

Cx37 has been shown to be capable of inhibiting the growth of endothelial and smooth muscle cells in vitro (FIG. 6B). Cx37 does not inhibit the growth of cell lines derived from other cell types. Specifically, Cx37 has no effect on NRK (normal rat kidney cell lines), N2A (rat neuroblastoma cell lines), melanoma cell lines, or rat breast cancer cell lines (FIG. 6A). Vascular endothelial and smooth muscle cells were very sensitive to Cx37-induced cell death, but others were resistant or not killed by Cx37.

Actively dividing endothelial cells were more susceptible to Cx37-induced apoptosis rather than quiescent, confluent endothelial cells (FIG. 6B). Cx37-induced cell death was also dose-dependent (FIG. 6B). Cx37 expression in actively dividing endothelial cells induced apoptosis and resulted in the death of cells within 3 days, but confluent, quiescent endothelial cells were resistant to the effects of Cx37. This indicates that Cx37 preferentially blocks new vessel formation, but leaves normal vessels intact. These show that the early stage of cell division is more susceptible to changed communication and it needs more precise control of intercellular communication through gap junction channels. It has been reported that during early stages of apoptosis and mitosis there was a relatively high level of gap junction intercellular communication. There are common features in early mitosis and early apoptosis, and strong signals to proliferate. Activation of cyclin-dependent kinases also promotes apoptosis. These suggest that keeping proper molecular filters with right connexins may be critical for cellular homeostasis of local environment.

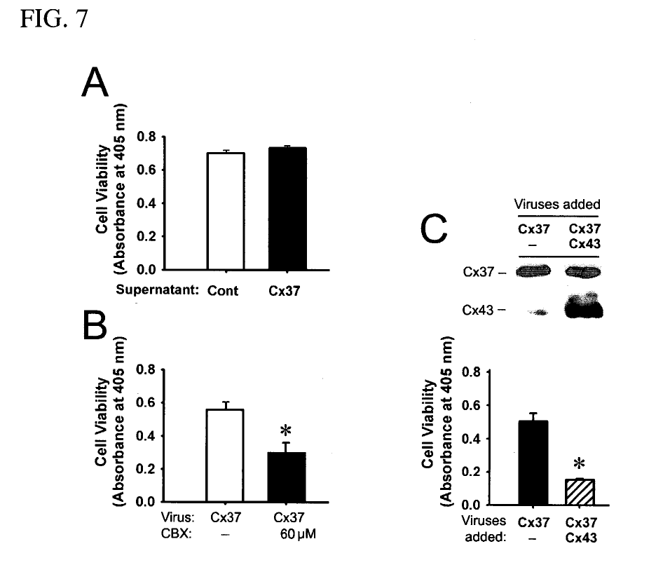

In another embodiment, antiangiogenetic effect of Cx37 can be potentiated in combination with other connexins and/or other molecules such as gap junction channel blockers (FIG. 7). In particular, overexpressed Cx43 (FIG. 7C) and Cx40 potentiated Cx37-induced cell death. Modification of Cx37-induced cell death with Cx43 is quite possible because Cx37 and Cx43 mix together in hemichannel and can make 12 different channels (complete gap junction channels) between adjacent cells. It has been shown that mixed channels of Cx37 and Cx43 show different electrophysiological and dye transfer characteristics in cultured cells. Based on the effects of Cx37 and Cx43 on HUVEC, gap junctions may play a regulatory role during initiation of these opposite yet equally important mechanisms of maintaining homeostasis. Carbenoxolone (known gap junction channel blocker) also potentiated Cx37-induced cell death (FIG. 7B).

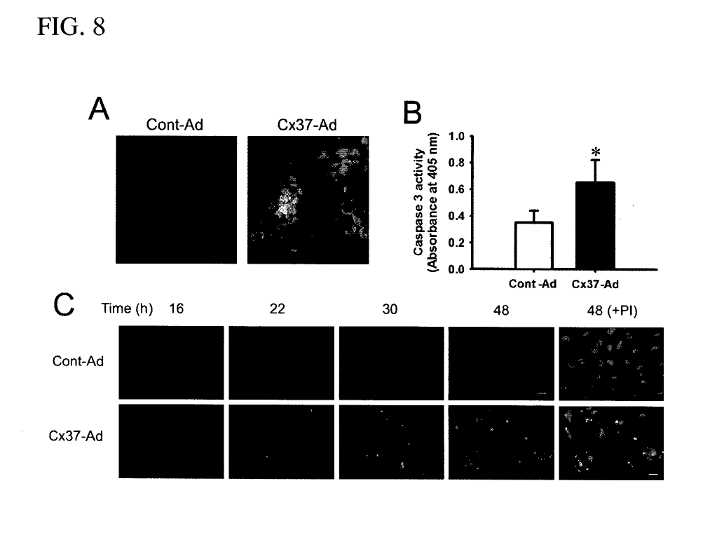

Cx37 kills HUVEC by inducing apoptosis (FIG. 8). In the controls, the cells showed intact endothelial cell morphology, whereas numbers of dying cells of Cx37-treated cells increased and detached from the culture plate. At the early stage of apoptosis, phosphatidylserines (PS) from the inner face of the plasma membrane were translocated to the cell surface. PS was detected with an FITC conjugated annexin V that binds naturally to PS (FIG. 8A). Caspase 3 is an intracellular protease activated early during apoptosis of mammalian cells and initiates cellular breakdown by degrading specific structural, regulatory, and DNA repair proteins. This enzyme was elevated in Cx37-treated cells (FIG. 8B). Fragmentation of nuclear DNA is one of the distinct morphological changes occurring in the nucleus of apoptotic cells. A TUNEL (TdT-mediated dUTP-X nick end labeling) assay was performed at different time points on Cx37 and control adenovirus-treated cells. Cx37-treated cells showed numerous positive cells, whereas no positive cells were seen in the control (FIG. 8C). Flow cytometry measured at 1 day of virus treatment shows that cell cycle profiles of Cx37 treated group have not been changed compared with those of control group, but after 2 days apoptotic cells of Cx37 virus group soared compared to those of control virus group (54.4±13.2 vs. 6.2±0.8, P<0.01).

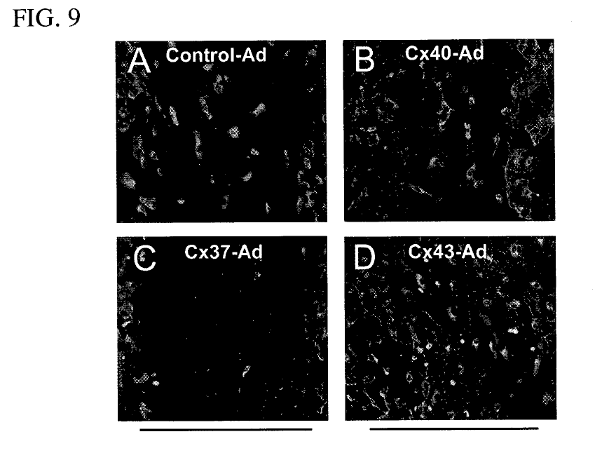

Connexins modulate migration of endothelial cells. To study how vascular connexins affect endothelial migrations, we used model of wound injury in cultured primary endothelial cells. Cx37 completely blocked the migration of endothelial cells from wounded edges. Cx43, followed by Cx40 in potency, significantly accelerated wound healing compared with control (FIG. 9). These results suggest that endothelial cells need appropriate gap junctions for their migration. The Cx43- and Cx40-adenovirus can also be utilized in many pathologic conditions to accelerate angiogenesis and thereby improve healing of wounded skin, damaged endothelial cells following balloon angioplasty, bone fractures, and skin graft.

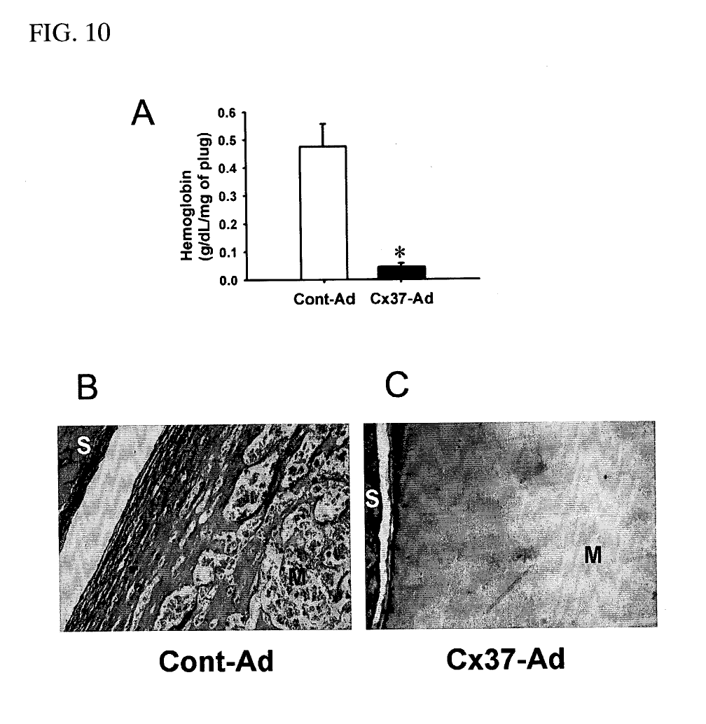

Cx37 blocks VEGF-induced angiogenesis in vivo (FIG. 10). To evaluate the in vivo effect of Cx37 on the formation on new capillaries, we performed a Matrigel plug assay in mice. We used vascular endothelial growth factor (VEGF), the most potent endogenous angiogenic factor, in order to induce angiogenesis into Matrigel. Systemic treatment as well as local delivery of Cx37-adenovirus into mice completely blocked new vessel formation into Matrigel as examined by Hematoxylin and Eosin staining of sectioned Matrigel, and by Hemoglobin assay of the Matrigel. These results show that Cx37 might block angiogenesis by inhibiting migration of endothelial cells from existing blood vessels. It may also induce regression of rapidly growing capillaries that is characteristic of solid tumors.

Gene Therapy

The present invention also encompasses gene therapy whereby the gene encoding connexins is regulated in a patient. Various methods of transferring or delivering DNA to cells for expression of the gene product protein, otherwise referred to as gene therapy, are disclosed in Gene Transfer into Mammalian Somatic Cells it vivo, N. Yang, Crit. Rev. Biotechn. 12(4): 335-356 (1992). Gene therapy encompasses incorporation of DNA sequences into somatic cells or germ line cells for use in either ex vivo or in vivo therapy. Gene therapy functions to replace genes, augment normal or abnormal gene function, and to combat infectious diseases and other pathologies.

Strategies for treating these medical problems with gene therapy include therapeutic strategies such as adding a functional gene to either replace the function of the defective gene or to augment a slightly functional gene; or prophylactic strategies, such as adding a gene for the product protein that will treat the condition or that will make the tissue or organ more susceptible to a treatment regimen. As an example of a prophylactic strategy, a gene such as connexin may be placed in a patient and thus prevent or promote occurrence of angiogenesis; or a gene that makes tumor vessels more susceptible to radiation could be inserted and then radiation of the tumor would cause increased killing of the tumor vessels and eventually the tumor cells.

Many protocols for transfer of connexin DNAs or connexin regulatory sequences are envisioned in this invention. Transfection of promoter sequences, other than one normally found specifically associated with connexin, or other sequences which would increase production of connexin protein are also envisioned as methods of gene therapy.

Gene transfer methods for gene therapy fall into three broad categories-physical (e.g., electroporation, direct gene transfer and particle bombardment), chemical (lipid-based carriers, or other non-viral vectors) and biological (virus-derived vector and receptor uptake). For example, non-viral vectors may be used which include liposomes coated with DNA. Such liposome/DNA complexes may be directly injected intravenously into the patient. It is believed that the liposome/DNA complexes are concentrated in the liver where they deliver the DNA to macrophages and Kupffer cells. These cells are long lived and thus provide long term expression of the delivered DNA. Additionally, vectors or the “naked” DNA of the gene may be directly injected into the desired organ, tissue or tumor for targeted delivery of the therapeutic DNA.

Gene therapy methodologies can also be described by delivery site. Fundamental ways to deliver genes include ex vivo gene transfer, in vivo gene transfer, and in vivo gene transfer. In ex vivo gene transfer, cells are taken from the patient and grown in cell culture. The DNA is transfected into the cells, the transfected cells are expanded in number and then reimplanted in the patient. In in vivo gene transfer, the transformed cells are cells grown in culture, such as tissue culture cells, and not particular cells from a particular patient. These “laboratory cells” are transfected, the transfected cells are selected and expanded for either implantation into a patient or for other uses.

In vivo gene transfer involves introducing the DNA into the cells of the patient when the cells are within the patient. Methods include using virally mediated gene transfer using a noninfectious virus to deliver the gene in the patient or injecting naked DNA into a site in the patient and the DNA is taken up by a percentage of cells in which the gene product protein is expressed. Additionally, the other methods described herein, such as use of a “gene gun,” may be used for in vitro insertion of connexin DNA or connexin regulatory sequences.

Chemical methods of gene therapy may involve a lipid based compound, not necessarily a liposome, to ferry the DNA across the cell membrane. Lipofectins or cytofectins, lipid-based positive ions that bind to negatively charged DNA, make a complex that can cross the cell membrane and provide the DNA into the interior of the cell. Another chemical method uses receptor-based endocytosis, which involves binding a specific ligand to a cell surface receptor and enveloping and transporting it across the cell membrane. The ligand binds to the DNA and the whole complex is transported into the cell. The ligand gene complex is injected into the blood stream and then target cells that have the receptor will specifically bind the ligand and transport the ligand-DNA complex into the cell.

Many gene therapy methodologies employ viral vectors to insert genes into cells. For example, altered retrovirus vectors have been used in ex vivo methods to introduce genes into peripheral and tumor-infiltrating lymphocytes, hepatocytes, epidermal cells, myocytes, or other somatic cells. These altered cells are then introduced into the patient to provide the gene product from the inserted DNA.

Viral vectors have also been used to insert genes into cells using in vivo protocols. To direct tissue-specific expression of foreign genes, cis-acting regulatory elements or promoters that are known to be tissue specific can be used. Alternatively, this can be achieved using in situ delivery of DNA or viral vectors to specific anatomical sites in vivo. For example, gene transfer to blood vessels it vivo was achieved by implanting in vitro transduced endothelial cells in chosen sites on arterial walls. The virus infected surrounding cells which also expressed the gene product. A viral vector can be delivered directly to the in vivo site, by a catheter for example, thus allowing only certain areas to be infected by the virus, and providing long-term, site specific gene expression.

Viral vectors that have been used for gene therapy protocols include but are not limited to, retroviruses, other RNA viruses such as poliovirus or Sindbis virus, adenovirus including helper-dependent or non-immunogenic adenoviral systems, adeno-associated virus, herpes viruses, SV 40, vaccinia and other DNA viruses. Replication-defective murine retroviral vectors are the most widely utilized gene transfer vectors. Murine leukemia retroviruses are composed of a single stranded RNA complexed with a nuclear core protein and polymerase (pol) enzymes, encased by a protein core (gag) and surrounded by a glycoprotein envelope (env) that determines host range. The genomic structure of retroviruses include the gag, pol, and env genes enclosed by the 5′ and 3′ long terminal repeats (LTR). Retroviral vector systems exploit the fact that a minimal vector containing the 5′ and 3′ LTRs and the packaging signal are sufficient to allow vector packaging, infection and integration into target cells providing that the viral structural proteins are supplied in traps in the packaging cell line. Fundamental advantages of retroviral vectors for gene transfer include efficient infection and gene expression in most cell types, precise single copy vector integration into target cell chromosomal DNA, and ease of manipulation of the retroviral genome.

The adenovirus is composed of linear, double stranded DNA complexed with core proteins and surrounded with capsid proteins. Advances in molecular virology have led to the ability to exploit the biology of these organisms to create vectors capable of transducing novel genetic sequences into target cells in vivo. Adenoviral-based vectors will express gene product proteins at high levels. Adenoviral vectors have high efficiencies of infectivity, even with low titers of virus. Additionally, the virus is fully infective as a cell free virion so injection of producer cell lines are not necessary. Another potential advantage to adenoviral vectors is the ability to achieve long term expression of heterologous genes in viva. “Leaky” viral gene expression from the vector itself results in the generation of anti-Ad cytotoxic T-lympocytes. This kind of immune response not only impacts the transgene expression in vivo but also results in significant acute inflammatory reactions in the host. Helper-dependent or non-immunogenic adenoviruses have been developed in order to eliminate such a major drawback of replication incompetent adenoviruses (first generation adenovirus). Viral promoter-driven vectors deliver target genes into another tissues as well as vascular cells. This may cause unwanted side effects. To eliminate this, vectors regulated by tissue-specific promoters such as endothelial- or smooth muscle-specific promoters have been developed.

Mechanical methods of DNA delivery include fusogenic lipid vesicles such as liposomes or other vesicles for membrane fusion, lipid particles of DNA incorporating cationic lipid such as lipofectin, polylysine-mediated transfer of DNA, direct injection of DNA, such as microinjection of DNA into germ or somatic cells, pneumatically delivered DNA-coated particles, such as the gold particles used in a “gene gun,” and inorganic chemical approaches such as calcium phosphate transfection. Another method, ligand-mediated gene therapy, involves complexing the DNA with specific ligands to form ligand-DNA conjugates, to direct the DNA to a specific cell or tissue.

It has been found that injecting plasmid DNA into muscle cells yields high percentage of cells, which are transfected and have sustained expression of marker genes. The DNA of the plasmid may or may not integrate into the genome of the cells. Non-integration of the transfected DNA would allow the transfection and expression of gene product proteins in terminally differentiated, non-proliferative tissues for a prolonged period of time without fear of mutational insertions, deletions, or alterations in the cellular or mitochondrial genome. Long-term, but not necessarily permanent, transfer of therapeutic genes into specific cells may provide treatments for genetic diseases or for prophylactic use. The DNA could be reinjected periodically to maintain the gene product level without mutations occurring in the genomes of the recipient cells. Non-integration of exogenous DNAs may allow for the presence of several different exogenous DNA constructs within one cell with all of the constructs expressing various gene products.

Particle-mediated gene transfer methods were first used in transforming plant tissue. With a particle bombardment device, or “gene gun,” a motive force is generated to accelerate DNA-coated high density particles (such as gold or tungsten) to a high velocity that allows penetration of the target organs, tissues or cells. Particle bombardment can be used in in vitro systems, or with ex vivo or in vivo techniques to introduce DNA into cells, tissues or organs.

Electroporation for gene transfer uses an electrical current to make cells or tissues susceptible to electroporation-mediated gene transfer. A brief electric impulse with a given field strength is used to increase the permeability of a membrane in such a way that DNA molecules can penetrate into the cells. This technique can be used in in vitro systems, or with ex vivo or in vivo techniques to introduce DNA into cells, tissues or organs.

Carrier mediated, gene transfer in vivo can be used to transfect foreign DNA into cells. The carrier-DNA complex can be conveniently introduced into body fluids or the bloodstream and then site specifically directed to the target organ or tissue in the body. Both liposomes and polycations, such as polylysine, lipofectins or cytofectins, can be used. Liposomes can be developed which are cell specific or organ specific and thus the foreign DNA carried by the liposome will be taken up by target cells. Injection of immunoliposomes that are targeted to a specific receptor on certain cells can be used as a convenient method of inserting the DNA into the cells bearing the receptor. Another carrier system that has been used is the asialoglycoportein/polylysine conjugate system for carrying DNA to hepatocytes for in vivo gene transfer.

The transfected DNA may also be complexed with other kinds of carriers so that the DNA is carried to the recipient cell and then resides in the cytoplasm or in the nucleoplasm. DNA can be coupled to carrier nuclear proteins in specifically engineered vesicle complexes and carried directly into the nucleus.

Gene regulation of connexins may be accomplished by administering compounds that bind to the connexin genes, or control regions associated with the connexin genes, or its corresponding RNA transcript to modify the rate of transcription or translation. Additionally, cells transfected with a DNA sequence encoding connexin genes may be administered to a patient to provide an iii vivo source of connexins. For example, cells may be transfected with a vector containing a nucleic acid sequence encoding connexins.

Therapeutic Composition

The connexin polypeptide or a combination of the connexin polypeptide with other connexin polypeptides or other compounds such as gap junction blockers of the present invention can be:

(i) administered systemically or locally to tumor-bearing humans or animals as anti-angiogenic therapy; or

(ii) administered systemically or locally to humans or animals that have angiogenesis-related disorders as angiogenic therapy

Connexin 37 polypeptide or connexin 37 polypeptide combined with other vascular connexins are effective in treating diseases or disease processes by inhibiting angiogenesis. The present invention includes a method of treating an angiogenesis mediated disease by administering a therapeutically effective amount of connexin polypeptides, or a biologically active fragment thereof, or different sets of combinations of connexin fragments that collectively possess anti-angiogenic or angiogenic activity. Angiogenesis mediated diseases include, but are not limited to, solid tumors; blood born tumors such as leukemias; tumor metastasis; benign tumors, for example hemangiomas, acoustic neuromas, neurofibromas, trachomas, and pyogenic granulomas; rheumatoid arthritis; psoriasis; ocular angiogenic diseases, for example, diabetic retinopathy, retinopathy of prematurity, macular degeneration, corneal graft rejection, neovascular glaucoma, retrolental fibroplasia, rubeosis; Osler-Webber Syndrome; myocardial angiogenesis; plaque neovascularization; telangiectasia; hemophiliac joints; angiofibroma; and wound granulation. Connexins are useful in the treatment of disease of excessive or abnormal stimulation of endothelial cells. These diseases include, but are not limited to, intestinal adhesions, Crohn's disease, atherosclerosis, scleroderma, and hypertrophic scars, i.e., keloids. Connexin can be used as a birth control agent by preventing vascularization required for embryo implantation. Connexins are useful in the treatment of diseases that have angiogenesis as a pathologic consequence such as cat scratch disease (Rochele minalia quintosa) and ulcers (Helicobacter pylori).

Connexin 43 polypeptide or connexin 43 polypeptide combined with other vascular connexins are effective in treating diseases or disease processes by promoting angiogenesis. The present invention includes the method of treating an angiogenesis mediated disease with an effective amount of connexin polypeptides, or a biologically active fragment thereof, or different sets of combinations of connexin fragments that collectively possess anti-angiogenic or angiogenic activity. The angiogenesis mediated diseases include, but are not limited to, restenosis after angioplasty, ischemic coronary artery disease, congestive heart failure, critical limb ischemia, and gastroduodenal ulcer.

Connexin polypeptides may be also used in combination with other compositions and procedures for the treatment of diseases. For example, a tumor may be treated conventionally with surgery, radiation or chemotherapy combined with the connexin polypeptide and then the connexin polypeptide may be subsequently administered to the patient to extend the dormancy of micrometastases and to stabilize and inhibit the growth of any residual primary tumor. Additionally, and in particular, connexin37, connexin40, connexin43, connexin45 or combinations thereof, may be combined with pharmaceutically acceptable excipients, and optionally sustained-release matrix, such as biodegradable polymers, to form therapeutic compositions.

A sustained-release matrix, as used herein, is a matrix made of materials, usually polymers, which are degradable by enzymatic or acid/base hydrolysis or by dissolution. Once inserted into the body, the matrix is acted upon by enzymes and body fluids. The sustained-release matrix desirably is chosen from biocompatible materials such as liposomes, polylactides (polylactic acid), polyglycolide (polymer of glycolic acid), polylactide co-glycolide (co-polymers of lactic acid and glycolic acid) polyanhydrides, poly(ortho)esters, polyproteins, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone. A preferred biodegradable matrix is a matrix of one of either polylactide, polyglycolide, or polylactide co-glycolide (co-polymers of lactic acid and glycolic acid).

The angiogenesis-modulating therapeutic composition of the present invention may be a solid, liquid or aerosol and may be administered by any known route of administration. Examples of solid therapeutic compositions include pills, creams, and implantable dosage units. The pills may be administered orally, the therapeutic creams may be administered topically. The implantable dosage units may be administered locally, for example at a tumor site, or which may be implanted for systemic release of the therapeutic angiogenesis-modulating composition, for example subcutaneously. Examples of liquid composition include formulations adapted for injection subcutaneously, intravenously, intraarterially, and formulations for topical and intraocular administration. Examples of aersol formulation include inhaler formulation for administration to the lungs.

The dosage of the connexin polypeptides of the present invention will depend on the disease state or condition being treated and other clinical factors such as weight and condition of the human or animal and the route of administration of the compound.

The connexin formulations include those suitable for oral, rectal, ophthalmic (including intravitreal or intracameral), nasal, topical (including buccal and sublingual), intrauterine, vaginal or parenteral (including subcutaneous, intraperitoneal, intramuscular, intravenous, intradermal, intracranial, intratracheal, and epidural) administration. The connexin formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described. The amino and carboxyl termini of connexins can be coupled to other molecules.

The present invention is not to be limited in scope by the specific embodiments described herein. Indeed, various modifications of the invention in addition to those described herein will become apparent to theose skilled in the art from the foregoing description and accompanying figures. Such modifications are intended to fall within the scope of the appended claims. The following examples are offered by way of illustration of the present invention, and not by way of limitation.

### EXAMPLES

### Example I

### Generation of Wild Type Vascular Cx37/Cx40/Cx43/Cx45 Adenoviral Recombinants

Cx37, Cx40, Cx43, and Cx45 were subcloned in pShuttle (Quantum Biotechnologies, Montreal, Canada), and epitopes such as HA or FLAG was attached to the carboxyl terminus of the coding regions to differentiate the expressed patterns from endogenous conlexins (as done by Larson et al., 2000). To generate recombinant adenovirus, we used Tile AdEasy™ System (Quantum Biotechnologies). We isolated several clones of each connexin recombinant to optimize connexin levels and to minimize toxicity due to viral proteins. Large-scale production of viral recombinant particles in QBI-293A cells was performed using selected clones of each connexin adenoviral recombinant.

The wild-type human nucleic acid sequence for Cx37 is represented in SEQ ID NO: 1 (corresponding amino acid sequence is represented by SEQ ID NO:2), which is discussed in Reed et al., J. Clin. Invest. 91 (3), 997-1004 (1993). A FLAG epitope having SEQ ID NO: 9 (corresponding to amino acid SEQ ID NO: 10) was attached to the end of cytoplasmic tail of human connexin37 to differentiate human Cx37 delivered by adenovirus from endogenous human Cx37. Anti-FLAG polyclonal antibodies (Sigma) were used for visualization of human Cx37-FLAG by immunohiostochemistry.

The wild-type mouse nucleic acid sequence for Cx40 is represented in SEQ ID NO:3 (corresponding amino acid sequence is represented by SEQ ID NO:4), which is discussed in Hennemann et al., J. Cell Biol. 117 (6), 1299-1310 (1992); and Seul et al., Genomics 46 (1), 120-126 (1997). A FLAG epitope having SEQ ID NO:9 (corresponding to amino acid SEQ ID NO:10) was attached to the end of cytoplasmic tail of connexin40 to differentiate Cx40 delivered by adenovirus from endogenous Cx40. Anti-Cx40 or anti-FLAG polyclonal antibodies (Sigma) were used for visualization of Cx40-FLAG by immunohistochemistry.

The wild-type mouse nucleic acid sequence for Cx43 is represented in SEQ ID NO:5 (corresponding amino acid sequence is represented by SEQ ID NO:6), which is discussed in Sullivan et al., Gene 130 (2), 191-199 (1993). A FLAG epitope having SEQ ID NO:9 (corresponding to amino acid SEQ ID NO:10) was attached to the end of cytoplasmic tail of connexin43 to differentiate Cx43 delivered by adeenovirus from endogenous Cx43. Anti-Cx43 or anti-FLAG polyclonal antibodies (Sigma) were used for visualization of Cx43-FLAG by immunohistochemistry.

The wild-type mouse nucleic acid sequence for Cx45 is represented in SEQ ID NO:7 (corresponding amino acid sequence is represented by SEQ ID NO:8), which is discussed in Sullivan et al., Gene 130 (2), 191-199 (1993). A HA epitope having SEQ ID NO:11 (corresponding to amino acid SEQ ID NO:12) was attached to the end of cytoplasmic tail of connexin45 to differentiate Cx45 delivered by adenovirus from Cx45. Anti-Cx45 or anti-HA polyclonal antibodies (Sigma) were used for visualization of Cx45-HA by immunohistochemistry.

The wild-type mouse nucleic acid sequence for Cx45 is represented in SEQ ID NO:7 (corresponding amino acid sequence is represented by SEQ ID NO:8), which is discussed in Sullivan et al., Gene 130 (2), 191-199 (1993). A HA epitope having SEQ ID NO:11 (corresponding to amino acid SEQ ID NO:12) was attached to the end of cytoplasmic tail of connexin45 to differentiate Cx45 delivered by adenovirus from Cx45. Anti-Cx45 or anti-HA polyclonal antibodies (Sigma) were used for visualization of Cx45-HA by immunohistochemistry.

### Example 2

### Generation of Mutant Type Vascular Cx37/Cx40/Cx43/Cx45 Adenoviral Recombinants

To eliminate responsive elements of cytoplasmic tail of Cx37, Cx40, Cx43, and Cx45 to protein kinases (protein kinase A & C), serine/threonine residues of cytoplasmic tails of vascular connexins were deleted, or mutated, or whole cytoplasmic tail were truncated.

### Example 3

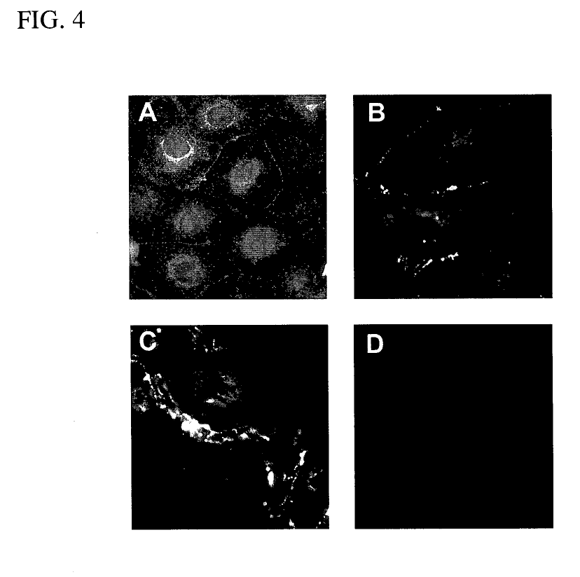

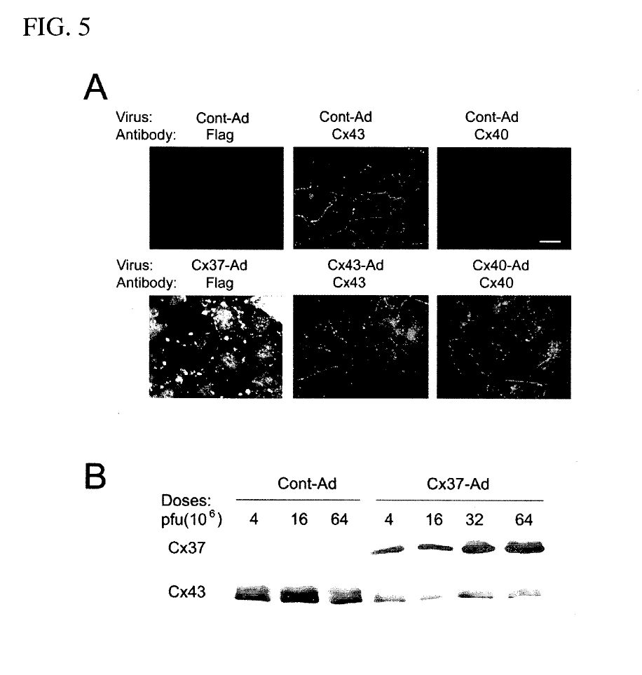

### Expression of Connexin Proteins in Cell Lines, Primary Endothelial Cells and Whole Animals by Connexin-Adenoviruses (FIGS. 4 and 5)

Each virus of 3.3×10⁷ pfu/well was treated confluent HUVEC grown in gelatin-coated 4 chamber slides (Nalge Nunc, Naperville, Ill.) for 1 day. Cells were immunostained with methods described previously. For Western blotting, confluent HUVEC grown in 100 mm culture dishes was harvested 1 day after virus treatment. Expression of Cx37 was measured as described previously. Antibodies against Cx37, Cx40, Cx43, Cx45, and antibody against FLAG and HA epitope (Sigma, St. Louis, Mo.) were used.

Normal human venous endothelial cells were purchased from Clonetics (Walkersville, Md., USA), and cultured in media supplemented with 10 ng/ml VEGF, 20 ng/ml basic FGF and 10 ng/ml EGF. All experiments were performed using subcultures between second and seventh passages. NRK and N2A cells were cultured in Dulbecco's modified Eagle's medium containing 5% fetal calf serum at 37° C. in an atmosphere of 5% CO₂. HUVEC were used in passages two through five. Contact-inhibited cells for more than 12 hours were used unless otherwise noted. Viruses were treated for 1 hour and changed with new media.

Connexins in adenoviral recombinants were successfully expressed in cells used. Immunostaining shows that connexins delivered by adenoviruses were localized to membranes at cell-cell contacts in a pattern of punctuate and linear staining (FIG. 4). The delivery of these connexins was dose-dependent (FIG. 5). Cultured HUVEC normally express connexin43 as a major interendothelial communications channel. No Cx37 staining could be detected among most cells and Cx40 was more abundant in cultured arterial endothelium than in cultured venous endothelium. Exogenous expression of Cx37 dramatically suppressed endogenous Cx43 expression in HUVEC. This suggests that cells regulate intercellular communication by regulating gap junction subtypes. Connexin proteins were successfully transferred into the cardiovascular system of adult animals. Connexins were expressed abundantly in all types of endothelial cells ranging from capillary, vein and artery (FIG. 4C).

### Example 4

### Cx37-Adenovirus Inhibits Proliferation of Cultured Human Umbilical Endothelial Cells (HUVECs) and Eventually Kills all of them in Higher Doses (FIG. 6)

To examine how Cx37 induce endothelial cell death, we performed assays for apoptosis. Results obtained from confluent cells used for these studies rule out possible involvement of Cx37 in cell cycle progress. Cells showing round shape appeared at around 1 and half days after treatment of Cx37, although it was variable by factor of doses of Cx37 and degree of confluency. In the controls, the cells showed intact endothelial cell morphology, whereas numbers of dying cells of Cx37-treated cells increased and were detached from the culture plate. At the early stage of apoptosis, phosphatidylserines (PS) from the inner face of the plasma membrane were translocated to the cell surface. PS was detected with an FITC conjugated annexin V that binds naturally to PS (FIG. 8A).

Caspase 3 is an intracellular protease activated early during apoptosis of mammalian cells and initiates cellular breakdown by degrading specific structural, regulatory, and DNA repair proteins. This enzyme was elevated in Cx37-treated cells (FIG. 8B). Fragmentation of nuclear DNA is one of the distinct morphological changes occurring in the nucleus of apoptotic cells. A TUNEL (TdT-mediated dUTP-X nick end labeling) assay was performed at different time points on Cx37 and control adenovirus-treated cells. Cx37-treated cells showed numerous positive cells, whereas no positive cells were seen in the control (FIG. 8C). Flow cytometry measured at one day after virus treatment showed that cell cycle profiles of Cx37 treated group had not been changed compared with those of control group, but after 2 days apoptotic cells of Cx37 virus group soared compared with those of control virus (54.4+13.2 vs. 6.2+0.8, P<0.01). These results strongly suggest that Cx37-induced endothelial cell death is mediated through apoptosis, although there are still possibilities of involvement of other mechanisms.

Without being limited by theory, it is believed that endogenous Cx43 and Cx40 have preference for survival signals rather than death signals. In normal conditions with endogenous Cx43 and Cx40, endothelial cells may be able to maintain their monolayers intact by exchanging survival signals through Cx43 and Cx40 channels preferentially. By experiments using cultured human umbilical endothelial cells (FIGS. 5-9, 12), this communication pattern may have changed when Cx37 channels began to be expressed and added on to the existing Cx43 channels and form Cx43/Cx37 heteromeric channels. We presume that channel preferences to intracellular molecules might have shifted from survival signals toward death signals when Cx37 become a dominant connexin among gap junction channels between cells. Death signals of apoptotic cells that exist in normal conditions can be a candidate. Signals within mitotic cells also can be a candidate if we consider actively dividing cells were more susceptible to Cx37-induced cell death rather than confluent ones.

Assay of cell proliferation. Cell proliferation was examined using XTT (sodium 3′-[1-(phenylaminocarbonyl)-3,4-tetrazolium]-bis(4-methoxy-6-nitro) benzene sulfonic acid hydrate) calorimetric assay (Roche Diagnostics).

Apoplosis assay. (a) Annexin V and propidium iodide (PI) staining of HUVEC. HUVEC cultured in gelatin-coated, 4 chamber slides were treated with 1.3×10⁸ pfu of control or Cx37 virus. After 1 and half days, apoptotic cells were visualized with Annexin V using Annexin-V-Fluos staining kit (Roche Diagnostics, Indianapolis, Ind.) and necrotic cells were with P1 without membrane permeabilization. (b) Caspase 3 assay. HUVEC in 100 mm culture dishes were treated with 2.1×10⁹ pfu of control or Cx37 virus. Cells were harvested and protein concentration was measured after 1 and half day's culture. Caspase activity was measured by using caspase 3 colorimetric activity assay kit (R&D Systems, Minneapolis, New England). (c) HUVEC cultured in gelatin-coated, 4 chamber slides were treated with 1.3×10⁸ pfu of control- or Cx37 virus at different times. Detection of fragmented DNA in HUVEC induced by Cx37 virus were performed with DeadEnd Fluorometric TUNEL system kit (Promega, Madison, Wis.), and all cells were stained with PI after membrane permeabilization. (d) Flow cytometry analysis. HUVEC in 100 mm dishes were treated with 4.1×10⁹ pfu of control or Cx37 virus. 1 or 2 days later, cells were trypsinized and stained with PI, and analyzed with a FACScan (Becton Dickinson, Franklin Lakes, N.J.) as described.

### Example 5

### Confluent Endothelial Cells are Resistant to Antiproliferative Effect of Cx37-Adenovirus (FIG. 6B)

Cx37 expression in actively dividing endothelial cells induced apoptosis and resulted in the death of cells within 3 days, but confluent, quiescent endothelial cells were resistant to this effect of Cx37. This strongly suggests that Cx37 preferentially blocks new vessel formation, but leaves normal vessels intact.

Susceptibility of actively dividing endothelial cells to Cx37-induced apoptosis indicates that Cx37 may be used as a new therapeutic tool in inhibiting new vessel growth from angiogenesis-related disorders. Cx43 effect on endothelial cell growth from wound edge indicates that Cx43 can be used in coronary angioplasty or other angiogenesis-related diseases. This provides new strategy in the control of angiogenesis: by targeting the regulation of angiogenesis at the level of transmission of intracellular signals.

### Example 6

Studies using cell lines of transformed endothelial cells have indicated that gap junctions are involved in wound healing of damaged vascular wall. To further determine how vascular connexins affect endothelial migrations, we used model of wound injury in cultured primary endothelial cells. Cx37 completely blocked the migration of endothelial cells from wounded edges. Cx43, followed by Cx40 in potency, significantly accelerated wound healing compared with control (FIG. 9). These results indicate that endothelial cells need appropriate gap junctions for their migration.

Wound repair assay. Confluent cells cultured in gelatin-coated culture dishes were incubated with adenoviral recombinants containing connexins for 24 h, and then the monolayers were mechanically wounded with the tip of a 10 ml pipette; detached cells were removed, and fresh complete medium was added. Cells migrating from wound edges were photographed using a Zeiss microscope (Zeiss).

### Example 7

### Cx37-Adenovirus Blocks Angiogenesis In Vivo.

VEGF-induced angiogenesis into Matrigel was blocked in adult mice. Systemic treatment as well as local delivery of Cx37-adenovirus into mice completely blocked new vessel formation into Matrigel as examined by Hematoxylin and Eosin staining of sectioned Matrigel (FIG. 7), and by Hemoglobin assay of the Matrigel (FIG. 10).

Animals. Seven-week old, specific pathogen-free male BALB/C mice (Charles River Laboratories, Wilmington, Mass.) were used for in vivo studies.

In vivo Matrigel Plug assay. (A) Local treatment with adenoviruses. Cx37- or control-adenovirus were mixed with 400 μl of Matrigel (BD Biosciences, Bedford, Mass.) supplemented with 50 ng/ml of VEGF, and 60 units of heparin. These adenovirus-mixed Matrigels were injected into mice subcutaneously. (B) Systemic treatment with adenoviruses. Cx37- or control-adenovirus was administered to mice through tail vein injection. 24 hours later, mice were given subcutaneous injections of 400 μl of Matrigel supplemented with only VEGF and heparin (no adenoviruses). After 7 days of Matrigel injection, mice were sacrificed and the Matrigel plugs were removed and fixed in 4% paraformaldehyde. The plugs were embedded in paraffin, sectioned, and H & E stained. Sections were examined by light microscopy, and photographed. Parts of Matrigels were used to measure hemoglobin content to quantitate angiogenesis induced by VEGF.

The anti-angiogenic effects of Cx37-adenovirus can be applied as a new therapeutic agent in many diseases, as examples, tumor growth and metastasis are angiogenesis-dependent. Unregulated angiogenesis may also result in different pathologies, such as rheumatoid arthritis, diabetic retinopathy, psoriasis and juvenile hemangiomas.

Currently, a large variety of chemotherapeutic drugs are being used to treat cancer. Unfortunately, many compounds have limited efficacy due to problems of delivery and penetration and due to limited selectivity for tumor cells, which potentially cause severe damage to healthy tissues. Tumor cells are a rapidly changing target because of their genetic instability, heterogeneity, and high rate of mutation, leading to selection and overgrowth of a drug-resistant tumor cell population.

Anti-angiogenic therapy, which targets activated endothelial cells, offers several advantages over therapy directed against tumor cells. Endothelial cells are a genetically stable, diploid, and homogenous target, and spontaneous mutations rarely occur. Because anti-angiogenic therapy is directed at activated endothelial cells, its target should be easily accessible by systemic administration. Different tumor cells are sustained by a single capillary. And tumor-associated endothelial cells contribute to both endothelial and tumor cell growth by releasing autocrine and paracrine factors. Consequently, the activated endothelium presents a more specific target than the tumor cells, and inhibition of a small number of tumor vessels may affect the growth of many tumor cells.

Angiogenesis is a complex process that includes endothelial cell proliferation, migration, and three-dimensional tube formation. In addition to in vitro evidences of connexins' involvement in endothelial growth and migration, blocking of VEGF-induced angiogenesis in mice strongly indicates that interendothelial communication through gap junctions is important in angiogenesis. This is the first evidence showing gap junctions can modulate angiogenesis of endothelial cells in humans and animals that are not genetically engineered.

### Example 8

### Effects of Combinations of Connexin and Modifiers of Gap Junction Channels on Angiogenesis (FIG. 7)

Experiments of conditioned media of Cx37-induced dead cells show that the death process spreads internally among cells rather than through extracellular routes (FIG. 7A). We blocked gap junction channels of HUVEC with carbenoxolone (known gap junction channel blocker), after 12 hours of Cx37 virus treatment. It potentiated Cx37-induced cell death (FIG. 7B), suggesting that cells are still trying to send survival signals over death signals through mixed Cx37/Cx43 channels. Absence of specific gap junction blockers to each connexins without cell toxicity made us unable to clarify the specific role of Cx37 and Cx43 during the course of cell death. Endogenous Cx43 has been down regulated by exogenous Cx37 (FIG. 5B), leaving questions that this may contribute to initiation of cell death.

To test this hypothesis, we overexpressed Cx43 in HUVEC with Cx43-adenovirus. Exogenous Cx43 expression with Cx43 adenovirus was not down regulated because it was driven by CMV promoter. Overexpressed Cx43 potentiated Cx37-induced cell death, (FIG. 7B). Modification of Cx37-induced cell death with Cx43 is quite possible because Cx37 and Cx43 mix together in hemichannel and can make 12 different channels (complete gap junction channels) between adjacent cells. It has been shown that mixed channels of Cx37 and Cx43 shows different electrophysiological and dye transfer characteristics in cultured cells. Based on effects of Cx37 and Cx43 on HUVEC, we propose that gap junctions may play a regulatory role during initiation of these opposite yet equally important mechanisms of maintaining homeostasis.

### Example 9

### Hematologic and Histologic Findings of Cx37-Adenovirus Treated Mice Systemically Support that Cx37 does not Affect Existing Adult Blood Vessels

We examined the possibility of disintegration of endothelial cells by Cx37 adenovirus in adult animals. There were no signs of leakage of red blood cells through capillaries or larger vessels macroscopically or histologically. We also examined RBCs and differential WBC counts in mice treated systemically with adenoviruses, and have found normal hematology findings in all of mice studied (data not shown). Within the 2 weeks of observation, we have not found any sign of abnormality between control and systemically treated mice.

Systemic delivery of connexin-adenovirus to adult blood vessels. Mice received tail vein injections of adenovirus containing connexins. Mice were sacrificed 5 or 7 days after tail vein injection of adenoviruses. Heart, aorta, lung, kidney, liver and skeletal muscle tissues were removed, and frozen-sectioned for immunohistochemical analysis of connexins.

### Example 10

In contrast to responses of endothelial cells to vascular connexins, non-endothelial cells, NRK and N2A showed different responses. NRK reacted with none of the connexins. N2A also did not respond to Cx37 or Cx40 (FIG. 6A). Contrary to Cx43 effects on HUVEC, Cx43 suppressed the growth of N2A cells (FIG. 6A). Once their intercellular communications have been restored by Cx43, the growth of N2A cells were slowed down.

### Example 11

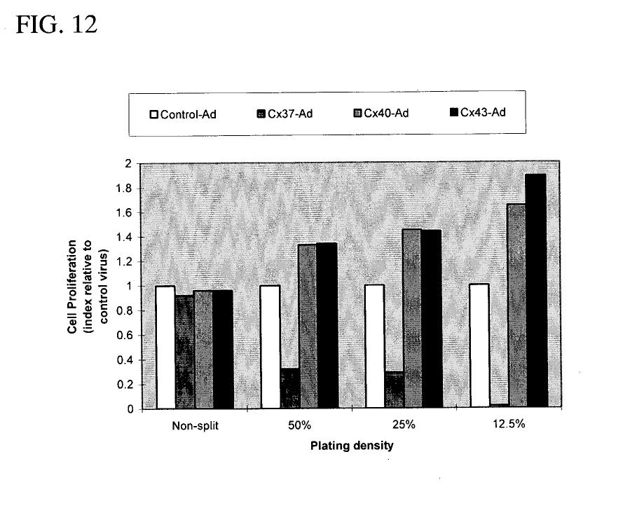

Small doses of connexins delivered by adenoviruses do not influence the proliferation of the confluent, quiescent endothelial cells, but they begin to influence cell proliferation when cells start to divide: Cx37 as an antiproliferative, and Cx43 and Cx40 as an proliferative intercellular molecular filter of endothelial cells (FIG. 12).

Small doses (less than 4×10⁷ pfu) of Cx37-adenovirus do not affect proliferation of confluent cells. This means that addition of a small amount of Cx37 channels between intercellular membranes is not adequate to disturb the balance between apoptotic and antiapoptotic signals that help confluent cells to maintain their overall cell populations. This might be possible by two ways; (1) another endogenous connexin (Cx43 and/or Cx40) in HUVECs may maintain the balances of the signals for cell proliferation, and/or (2) types of intracellular signals passing through gap junctions in the confluent endothelial cells are different from those of actively dividing cells, and then their transfer ratio between cells through gap junctions were less affected by small quantity of exogenous Cx37.

What happens when adenovirus-connexin treated endothelial cells are exposed to mitotic signals and begin to divide? Control cells regulate the level of endogenous connexins and therefore the proliferation signals that pass through gap junction channels during the course of the cell cycle. These cells control only native intracellular signals during the course of a cell cycle, and communicate actively with neighboring cells through gap junctions more at the beginning stage of cell cycle than the late stage. Such cell cycle-dependent regulation of gap junction mediated intercellular communication is abolished when gap junction genes linked to a viral promoter in an adenoviral vector are administered to the cells. In particular, without being limited to any particular vector, in an adenovirus-connexin treated endothelial cell, the regulatory elements of recombinant adenovirus control the expression of exogenously introduced connexin molecules. Viral regulatory elements express connexins regardless of the stage of cell cycle in the host cell. This indicates that the fate of a cell may be changed even if the same type of connexins exist in the host cell, if the connexin is expressed constantly regardless of the stage of its cell cycle. Moreover, the impact of such constant expression of connexin on cell cycle will be significant if different types of connexins are delivered to host cells by a vector that constantly expresses the connexin gene, such as a virus, and in particular, adenovirus.

We tested this hypothesis through the experiment shown in FIG. 12. We found that Cx37 still had a significant potential for antiproliferative effect, and that Cx43 and Cx40 have stimulating effects on cell proliferation. Cx43 and Cx40 (Cx43>Cx40) strongly stimulated the migration of endothelial cells from wound edges (FIG. 9) and did not increase apoptosis.

Angiogenesis is a fundamental process in reproduction and wound healing. Angiogenic effects of Cx43- and Cx40-adenovirus can also be used in many pathologic conditions, such as for improving healing of wounded skin, damaged endothelial cells after balloon angioplasty, bone fracture, and skin graft by accelerating angiogenesis.

### REFERENCES

Beyer E C, Seul K H, Larson D M. Cardiovascular gap junction proteins: molecular characterization and biochemical regulation, in Heart Cell communication in Health and Disease, edited by DE MELLO W C, JANSE M J, Norwell, M A, Kluwer Acadeiiic Publishers, p. 45-51 (1997).

Beyer E C, Willecke K. Gap junction genes and their regulation. Adv. Mol. Cell Biol. 30:1-30 (2000).

Blackburn J P, Peters N S, Yeh H I, Rothery S, Green C R, Severs N J. Upregulation of connexin43 gap junctions during early stages of human coronary atherosclerosis. Arterioscler. Thromb. Vase. Biol. 15(8): 1219-28 (1995).

Cai W J, Koltai S, Kocsis E, Scholz D, Schaper W, Schaper J. Connexin37, not Cx40 and Cx43, is induced in vascular smooth muscle cells during coronary arteriogenesis. J. Mol. Cell. Cardiol. 33(5): 957-67 (2001).

Davis L M, Kanter H L, Beyer E C, Saffitz J E. Distinct gap junction phenotypes in cardiac tissues with disparate conduction properties. J. Am. Coll. Cardiol. 24:1124-1132 (1994).

de Wit C, Roos F, Bolz S S, Kirchhoff S, Kruger O, Willecke K, Pohl U. Impaired conduction of vasodilation along arterioles in connexin40-deficient mice. Circ. Res. 31; 86(6): 649-55 (2000).

Edwards G, Feletou M, Gardener M J, Thollon C, Vanhoutte P M, Weston A H. Role of gap junctions in the responses to EDHF in rat and guinea-pig small arteries. Br. J. Phatiacol. 128(8): 1788-94 (1999).

Folkman J. Angiogenesis in cancer, vascular, rheumatoid and other disease. Nat Med 1:27-31 (1995).

Hyder S M and Stancel G M. Regulation of angiogenic growth factors in the female reproductive tract by estrogens and progestins. Mol Endocrinol 13:806-811 (1999).

Kwak B R, Pepper M S, Gros D B, Meda P. Inhibition of endothelial wound repair by dominant negative connexin inhibitors. Mol. Biol. Cell 12(4): 831-45 (2001). Larson D M, Seul K H, Berthoud V M, Lau A F, Sagar G D, Beyer E C. Functional Expression and Biochemical Characterization of an Epitope-Tagged Connexin37. Mol. Cell. Biol. Res. Commun. 3(2): 115-121 (2000).

Larson D M, Wrobleski M J, Sagar G D, Westphale E M, Beyer E C. Differential regulation of connexin43 and connexin37 in endothelial cells by cell density, growth, and TGF-beta1. Am. J. Physiol. 272(2 Pt 1): C405-15 (1997).

Liao Y, Day K H, Damon D N, Duling B R. Endothelial cell-specific knockout of connexin 43 causes hypotension and bradycardia in mice. Proc. Natl. Acad. Sci. U S A 14; 98(17):9989-94 (2001).

Liekens S, De Clercqa E, Neytsa J. Angiogenesis: regulators and clinical applications. Biochemical Pharmacology 61:253-270 (2001)

McCormick S M, Eskin S G, McIntire L V, Teng C L, Lu C M, Russell C G, Chittur K K. DNA microarray reveals changes in gene expression of shear stressed human umbilical vein endothelial cells. Proc. Natl. Acad. Sci. USA. 98(16): 8955-60 (2001).

Seul K H and Beyer E C. Heterogeneous localization of connexin40 in the renal vasculature. Microvasc Res. 59(1): 140-8 (2000).

Thompson W D, Li W W, Maragoudakis M. The clinical manipulation of angiogenesis: pathology, side-effects, surprises, and opportunities with novel human therapies. J Pathol 187: 503-510 (1999).

Yasui K, Kada K, Hojo M, Lee J K, Kamiya K, Toyama J, Opthof T, Kodaina I. Cell-to-cell interaction prevents cell death in cultured neonatal rat ventricular myocytes. Cardiovasc Res 48(1):68-76 (2000)

Yeh H I, Lai Y J, Chang H M, Ko Y S, Severs N J, Tsai C H. Multiple connexin expression in regenerating arterial endothelial gap junctions. Arterioscler. Thromb. Vase. Biol. 20(7): 1753-62 (2000).

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

What is claimed is:

1. A method of modulating angiogenesis comprising administering a gap junction polypeptide to endothelial cells.

2. The method according to claim 1, wherein said gap junction polypeptide is vascular connexin polypeptide.

3. The method according to claim 2, wherein said connexin is connexin37 (Cx37), connexin40 (Cx40), connexin43 (Cx43), connexin45 (Cx45) or combination thereof.

4. A method of modulating angiogenesis comprising
a) generating a recombinant viral or plasmid vector comprising a DNA sequence encoding a member of a connexin family of polypeptides operatively linked to a promoter;
b) transfecting in vitro a population of cultured cells with said recombinant vector, resulting in a population of transfected cells; and
c) transplanting said transfected cells to a mammalian host, such that expression of said DNA sequence within the mammal results in modulating angiogenesis.

5. A method of inhibiting proliferation of endothelial cells comprising administering to the endothelial cells connexin37 polypeptide or a variant thereof.

6. A method of inhibiting growth or proliferation of endothelial cells, comprising:
a) generating a recombinant viral or plasmid vector comprising a DNA sequence encoding a member of a connexin family of polypeptides operatively linked to a promoter;
b) transfecting in vitro a population of cultured cells with said recombinant vector, resulting in a population of transfected cells; and
c) transplanting said transfected cells to a mammalian host, such that expression of said DNA sequence within the mammal results in inhibition of endothelial cell growth or proliferation.

7. The method according to claim 6, wherein the vector is viral vector.

8. The method according to claim 6, wherein the vector is plasmid vector.

9. The method according to claim 6, wherein the connexin is connexin37.

10. A method of promoting growth or proliferation of endothelial cells, comprising administering to the endothelial cells a connexin polypeptide or a variant thereof.

11. The method of claim 10, wherein the connexin polypeptide is connexin40, connexin43 or connexin45 polypeptide or a variant thereof.

12. A method of promoting growth or proliferation of endothelial cells, comprising:
a) generating a recombinant viral or plasmid vector comprising a DNA sequence encoding a member of a connexin family of polypeptides operatively linked to a promoter;
b) transfecting in vitro a population of cultured cells with said recombinant vector, resulting in a population of transfected cells; and
c) transplanting said transfected cells to a mammalian host, such that expression of said DNA sequence within the mammal results in promotion of endothelial cell growth or proliferation.

13. The method according to claim 12, wherein the vector is viral vector.

14. The method according to claim 12, wherein the vector is plasmid vector.

15. The method according to claim 12, wherein the connexin is connexin40, connexin43, connexin45 or a combination thereof.

16. A method of treating angiogenesis related disease comprising administering to a mammal in need thereof a therapeutically effective amount of a gap junction polypeptide.

17. The method according to claim 16, wherein the gap junction polypeptide is connexin37 (Cx37), connexin40 (Cx40), connexin43 (Cx43), connexin45 (Cx45) or a combination thereof.

18. The method according to claim 16, wherein the angiogenesis related disease is solid tumors, blood born tumors, tumor metastasis, benign tumors, rheumatoid arthritis, psoriasis, ocular angiogenic diseases, Osler-Webber Syndrome, myocardial angiogenesis, plaque neovascularization, telangiectasia, hemophiliac joints, angiofibroma, wound granulation, intestinal adhesions, Crohn's disease, atherosclerosis, scleroderma, or hypertrophic scars.
